(19) United States
(12) Patent Application Publication
(10) Pub. No.: US 2016/0022123 A1
(43) Pub. Date: Jan. 28, 2016

(54) MANEUVERING COILS SETUP FOR MANEUVERING A SWALLOWABLE IN-VIVO DEVICE

(71) Applicant: GIVEN IMAGING LTD., Yoqneam (IL)

(72) Inventors: Ehud KATZNELSON, Ramat Yishay (IL); Haim ROTEM, Mate Asher (IL); Eshel HASON, Yoqneam IIit (IL)

(21) Appl. No.: 14/774,715

(22) PCT Filed: Mar. 11, 2014
(86) PCT No.: PCT/IL14/50250

§ 371 Date: Sep. 11, 2015

Related U.S. Application Data
(60) Provisional application No. 61/775,977, filed on Mar. 11, 2013.
(60) Provisional application No. 61/917,770, filed on Dec. 18, 2013.

(51) Int. Cl. A61B 1/00; A61B 1/04; H01F 7/20

(52) U.S. Cl. CPC A61B 1/00158; H01F 7/202; H01F 7/204; A61B 1/041; A61B 2019/2261

(57) ABSTRACT

A ternary coil assembly (TCA) is provided, which may include an anterior coil, a posterior coil adjacently mounted side by side with respect to, and electrically isolated from and forming a plane with, the anterior coil, and an ancillary coil encircling, and electrically isolated from, the anterior and posterior coils. A magnetic field maneuvering system may include a number N of TCAs that may be positioned circularly. The circularly positioned TCAs, by manipulating their electrical current, may be operated to generate a magnetic field maneuvering pattern (MMP) such that a magnetic field may be generated in a first direction to orient a device in that direction, and a magnetic field gradient in a second direction to apply a movement force in the second direction. The direction of the magnetic field and the direction of the magnetic field gradient may differ, that is, they may be controlled independently.

### FIELD OF THE INVENTION

The present invention generally relates to maneuvering of a magnet in a magnetic field and more specifically to magnetic field based maneuvering system and methods for maneuvering a swallowable in-vivo device, and to a ternary coil assembly serving as a building block of the magnetic maneuvering system.

### BACKGROUND

In-vivo measuring systems are known in the art. Some in-vivo devices/systems, which may traverse the gastrointestinal (“GI”) system, or other body organs/systems, may include an imaging sensor, or imager, for imaging (e.g., capturing images of) the interior of the GI system. An in-vivo device may include one or more imagers. Other in-vivo devices may alternatively or additionally include a medication container and means for administering medication in the GI system. Other in-vivo devices may include means for performing surgical operations in vivo, and so on.

Autonomous in-vivo devices are devices that traverse the GI system by being pushed through the GI system by peristaltic force exerted by the digestive system. Autonomous in-vivo devices may also spasmodically move in the intestinal tract in ‘fits and starts’. Moving a device in vivo by using a peristaltic force has drawbacks. For example, the in-vivo device may get stuck somewhere in the GI system for an unknown period of time; the device may capture images in one direction while a nearby area, which may be clinically more interesting, is not imaged sufficiently or at all. In addition, due to the length of the intestinal tract (several meters), it takes an in-vivo device several hours to traverse the entire GI system. In order to minimize discomfort to a patient and to allow her/him to have as normal life as possible during that time, the patient is asked to wear a data recorder for recording the images captured in vivo, in order for them to be analyzed at a later stage (e.g., after the in-vivo device is finally pushed out of the GI). When a physician reviews the images, or a selection thereof, s/he cannot be certain that all the clinically interesting, or intended, areas of the GI system were imaged.

Due to the anatomically-inhomogeneous nature of the GI system—it has anatomically distinct sections such as the small bowel and the colon—and/or to different susceptibility of its various sections to diseases, indiscriminately handling large number of images and frames by the in-vivo device is oftentimes superfluous. In part, this is because relatively less susceptible areas of the intestinal tract are overly imaged. More susceptible areas of the intestinal tract, on the other hand, may be imaged sparingly. The number of images captured from susceptible areas of the intestinal tract may be smaller than clinically desired. It may often be desirable to examine only one specific part of the GI tract, for example, the small bowel (“SB”), the colon, gastric regions, or the esophagus.

There exist magnetic maneuvering systems for maneuvering in-vivo devices magnetically. A device may be maneuvered magnetically by incorporating a magnet in it. Such maneuvering systems typically generate a magnetic field that aligns or moves the magnetic moment of the device magnet in the direction of the applied magnetic field, and moves the in-vivo device in a direction of a magnetic gradient whose direction is also aligned or positioned in the same direction as the direction of the magnetic field. With both the magnetic field and magnetic gradient aligned in the direction of the magnetic field, maneuverability of devices is limited.

While moving an in-vivo device through the GI is beneficial, there are some drawbacks associated with autonomous in-vivo devices in the GI tract. It would be beneficial to have a full control over such movement, including maneuvering the in-vivo device to a desired location and/or orientation and/or angular position or state in the GI system, or other body organ, and maintaining the location/orientation/angular position or state for as long as required or needed, for example to take additional pictures of a site and/or to release medication(s) in the site, or to move the in-vivo device in a wanted path/route.

### SUMMARY

A ternary coil assembly (“TCA”) is provided, which may include an anterior coil, a posterior coil adjacently mounted side by side with respect to, and electrically isolated from, and forming a plane with, the anterior coil, and an ancillary coil that may be attached to, or encircle, and electrically isolated from, the anterior and posterior coils. (The term “ternary coil assembly” refers herein to a magnetic coil structure including three coils which are tightened together.) A magnetic field generating coils setup, or magnetic system, may include a number N of TCAs that may be positioned circularly. The circularly positioned TCAs, by manipulating their electrical current, may be operated to generate a magnetic field maneuvering pattern (“MMP”) such that a magnetic field, which is part or component of the MMP, may be generated in a first direction to orient a magnetic device in that direction, and a magnetic field gradient, which is another part or component of the MMP, to generate magnetic force in a second direction to apply a magnetic force in a direction different than the first direction (e.g., in the second direction). The direction of the magnetic field (the first direction) and the direction of the magnetic field gradient (e.g., the second direction) may differ, as they may be controlled independently.

Some embodiments may include optimization of an electrical power that may be provided to the N ternary coil assemblies, where the optimization process may include selecting TCAs, and/including their currents, such that the selected TCAs jointly generate the required MMP with as minimal power as possible.

### BRIEF DESCRIPTION OF THE DRAWINGS

Various exemplary embodiments are illustrated in the accompanying figures with the intent that these examples not be restrictive. It will be appreciated that for simplicity and clarity of the illustration, elements shown in the figures referenced below are not necessarily drawn to scale. Also, where considered appropriate, reference numerals may be repeated among the figures to indicate like, corresponding or analogous elements. Of the accompanying figures:

FIG. 1 illustrates a conjugated coils assembly/setup according to an embodiment of the invention;

FIG. 2 illustrates a pair of conjugated coils assembly/setup according to an embodiment of the invention;

FIG. 3 illustrates a ternary coils assembly according to an example embodiment of the invention;

FIG. 4 illustrates eight ternary coils assemblies spread out according to an example embodiment of the invention;

FIG. 5 illustrates eight, circularly positioned, ternary coils assemblies positioned according to an example embodiment of the invention;

FIGS. 6A-6F depict components of a ternary coil assembly and a ternary coil assembly according to an example embodiment of the invention;

FIG. 7A depicts circularly positioned ternary coil assemblies setup according to an example embodiment of the invention;

FIG. 7B depicts a maneuvering coils system including the circular ternary coil assemblies setup of FIG. 7A and Z-coils according to an example embodiment of the invention;

FIG. 8A is a block diagram of a magnetic maneuvering system according to an example embodiment of the invention;

FIG. 8B is a block diagram of a magnetic maneuvering control system according to an example embodiment of the invention;

FIG. 9 is a block diagram of an in-vivo system according to an example embodiment of the invention;

FIG. 10 shows a magnetically maneuvering method according to an example embodiment of the invention;

FIG. 11 shows a magnetically maneuvering method according to another example embodiment of the invention;

FIGS. 12A-12F depict a TCA according to another example embodiment of the present invention; and

FIGS. 13A-13B show maneuvering coils setup according to another example embodiment of the invention.

### DETAILED DESCRIPTION

The description that follows provides various details of exemplary embodiments. However, this description is not intended to limit the scope of the claims but instead to explain various principles of the invention and the manner of practicing it.

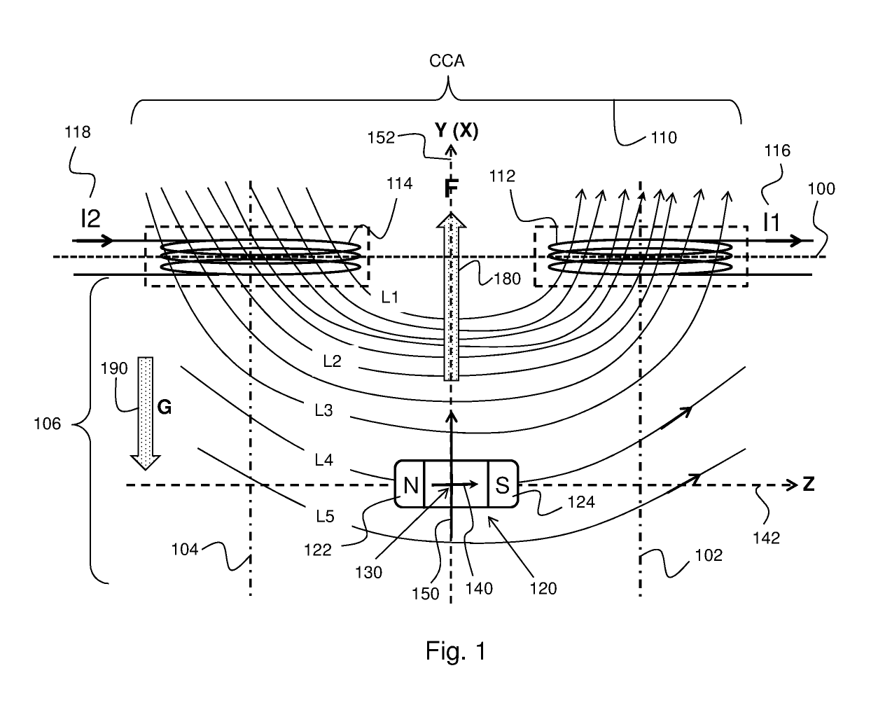

The magnetic field maneuvering system described herein is capable of determining the direction of the magnetic field independently of the direction of the magnetic field gradient. That is, depending on the circumstances (e.g., intended/next device's location and/orientation vis-a-vis the current device's location and/orientation), the direction of the magnetic field and the direction of the magnetic field gradient can differ to maximize maneuverability of the maneuvered device (e.g., in-vivo device). Separation between the two directions may be done, for example, by ‘bending’ the magnetic field, for example, by using conjugated coils that are adjacently positioned side by side on the same plane, as shown in the drawings, for example in FIGS. 1, 2, 3, 6E, and 6F. (The term “conjugated coils assembly/setup” refers to a magnetic coil structure where two magnetic coils lie adjacently side by side.) Some embodiments are described herein as including, or using, eight TCAs. However, other numbers of TCAs may be used; e.g., less than eight TCAs (e.g., two TCAs, three TCAs, four TCAs, etc.), or more than eight TCAs (e.g., nine TCAs, ten TCAs, etc.)

FIG. 1 illustrates a conjugated coils assembly/setup 110 according to an embodiment of the invention. Conjugated coils setup 110 may include an anterior coil 112 and a posterior coil 114. The anterior and posterior coils may adjacently be mounted side by side on a common plane 100. (The relative terms ‘anterior’ and ‘posterior’, as used herein, designate relative locations in the Z direction (e.g., on the Z axis). (‘Anterior’ may designate or be a leading coil and ‘posterior’ may designate or be a trailing coil with respect to the Z direction or axis.) Anterior coil 112 and posterior coil 114 may respectively have an anterior axis 102 and a posterior axis 104 that may be parallel to anterior axis 102. Each of the anterior and posterior coils (112,114) may be configured to individually generate a magnetic field in a direction aligned with the respective axis and, jointly, in a direction that may be (but not necessarily) perpendicular to anterior and posterior axes (102,104). For example, the magnetic field jointly generated by anterior and posterior coils (112,114) may be in direction Z (direction Z is shown at 142).

The direction of the magnetic field (some lines of which are shown at ‘L1’, ‘L2’, ‘L3’, and so on) at any particular spatial point within an operating region/space 106 may controllably be changed as a function of the strength and direction of electrical currents I1 and I2 (I1 and I2 are respectively shown at 116 and 118) that respectively flow through coils 112 and 114. The magnetic field gradient and the direction of the magnetic field gradient may also depend on the strength and direction of electrical currents I1 and I2. By way of example, a permanent magnet 120 is shown located at point 130, where the magnetic field (magnetic line L4) is aligned in direction 140 (coinciding with direction Z) and the magnetic field gradient is aligned in direction 150 that is perpendicular to the direction 140 of the magnetic field at that point. Permanent magnet 120 has a ‘north’ pole 122 and a ‘south’ pole 124, and the magnetic moment of permanent magnet 120 is aligned by the magnetic field at point 130 in the Z direction.

Posterior coil 114 and anterior coil 112 create magnetic field in the Z direction that has a non-zero magnetic field gradient

$$\frac{\partial {B}_{z}}{\partial Y} ,$$

as illustrated by the changing density in magnetic lines L1-L5 (e.g., the gradient becomes denser in direction Y. The field gradient creates force 180 on magnet 120 in the Y direction, and this magnetic force may counteract gravity force 190. The strength and direction of electrical currents I1 and I2 in the anterior coil and posterior coil, respectively, may be manipulated, for example, such that magnet 120 may be moved upwards/vertically (e.g., in direction 150), or slantingly (at an angle) with respect to the parallel axes of anterior coil 112 and posterior coils 114 (or with respect to the Z direction).

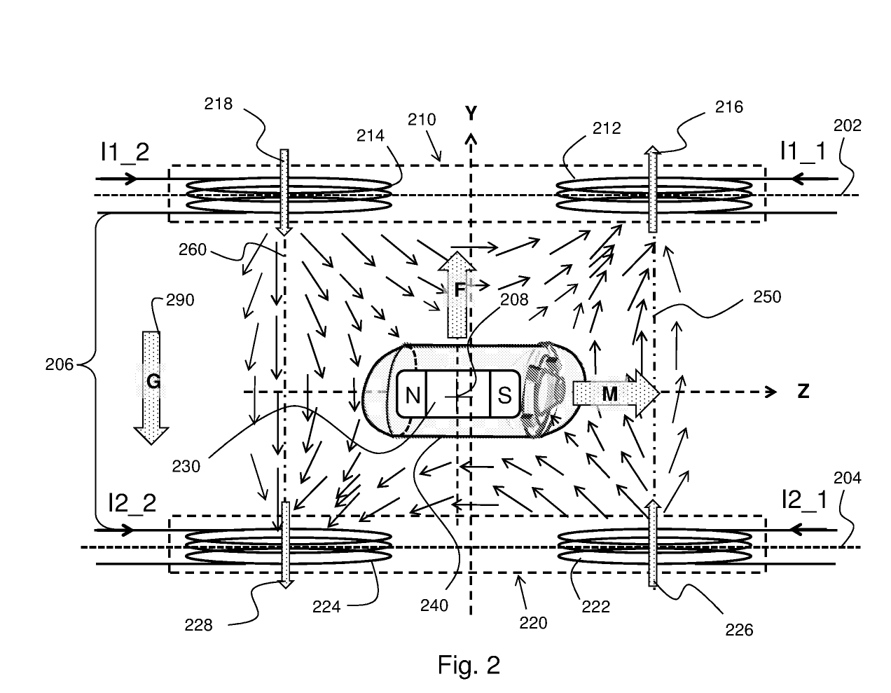

FIG. 2 schematically illustrates a pair of conjugated coils assemblies (“CCAs”)/setup (210,220) according to an example embodiment of the invention. CCA 210 may include an anterior coil 212 and a posterior coil 214. Anterior coil 212 and posterior coil 214 may adjacently be mounted side by side such that they form, or lie on, a common plane 202. Anterior coil 212 and posterior coil 214 may, respectively, have an anterior axis 250 and a posterior axis 260 that may be parallel to anterior axis 250. CCA/setup 220 may include an anterior coil 222 and a posterior coil 224. Anterior coil 222 and posterior coil 224 may adjacently be mounted side by side such that they form, or lie on, a common plane 204. Anterior coil 222 may have an anterior axis that may coincide with anterior axis 250, and posterior coil 224 may have a posterior axis that may coincide with posterior axis 260.

CCA 210 and CCA 220 may structurally be parallel (e.g., planes 202 and 204 may be parallel) or at an angle with respect to each other. By saying that CCA 210 and CCA 220 are structurally parallel is meant, in some embodiments, that anterior coil 212 is parallel to anterior coil 222 and posterior coil 214 is parallel to posterior coil 224. CCA 210 and CCA 220 may structurally overlap partly or completely. For example, anterior coil 212 may completely overlap anterior coil 222 but posterior coil 214 may only partly overlap posterior coil 224, or vice versa, or both anterior coil 212 and posterior coil 214 may respectively overlap anterior coil 222 and posterior coil 224 only partly.

Anterior coils 212 and 222 and posterior coils 214 and 224 may be configured to individually generate a magnetic field in a direction aligned with the respective axis, and jointly in a direction that may be (but not necessarily) perpendicular to the anterior and posterior axes (250,260). For example, the magnetic field jointly generated by coils 212, 214, 222 and 224 may be in the Z direction or in any direction which is at an angle relative to the Z direction.

The direction of the magnetic field at a particular spatial point within an operating region/space 206 may change as a function of the strength and direction of electrical currents I1_1, I1_2, I2_1 and I2_2 that respectively flow through coils 212, 214, 222 and 224. The magnetic gradient, including its direction, may also depend on the strength and direction of these currents. By way of example, a permanent magnet 230 is shown included in an example in-vivo device 240 that is located at spatial point 208. Permanent magnet 230 may have a ‘north’ (N) pole and a ‘south’ (S) pole, with a magnetic moment, M, that is in the Z direction.

Assuming that the magnetic field generated by CCA 210 and CCA 220 in the operating region/space 206 is symmetrical, which means that the intensity of the magnetic field at the center (e.g., at point 208) is theoretically zero. This means that there is no net force (F) on magnet 230 in any direction. Therefore, magnet 230, and therefore in-vivo device 240, does not move from (it is retained to) point 208.

As explained above in connection with FIG. 1, using only one conjugated coils assembly (CCA) would result in unstable system in terms of movement controllability. However, addition of a second, ‘counter’ or opposite, CCA enables stabilizing the magnetic movement controllability because the second CCA enables generating restraining forces that stabilize the magnet's movement controllability. For example, the magnetic force applied to magnet 230 may be controlled to levitate in-vivo device 240 against gravity force 290 at any desired {X,Y,Z} point within operating region/space 206.

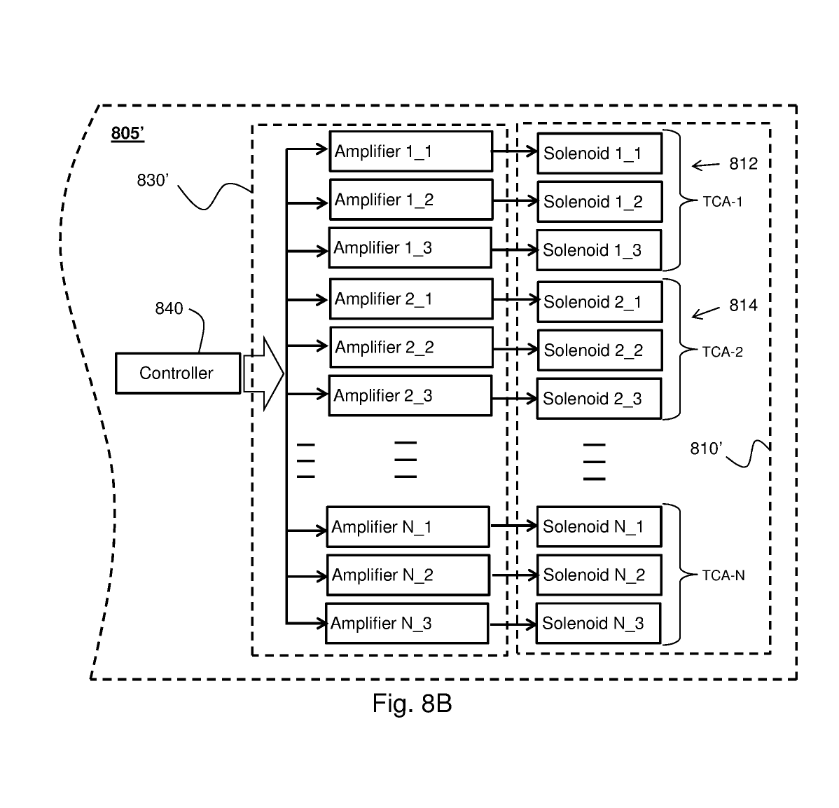

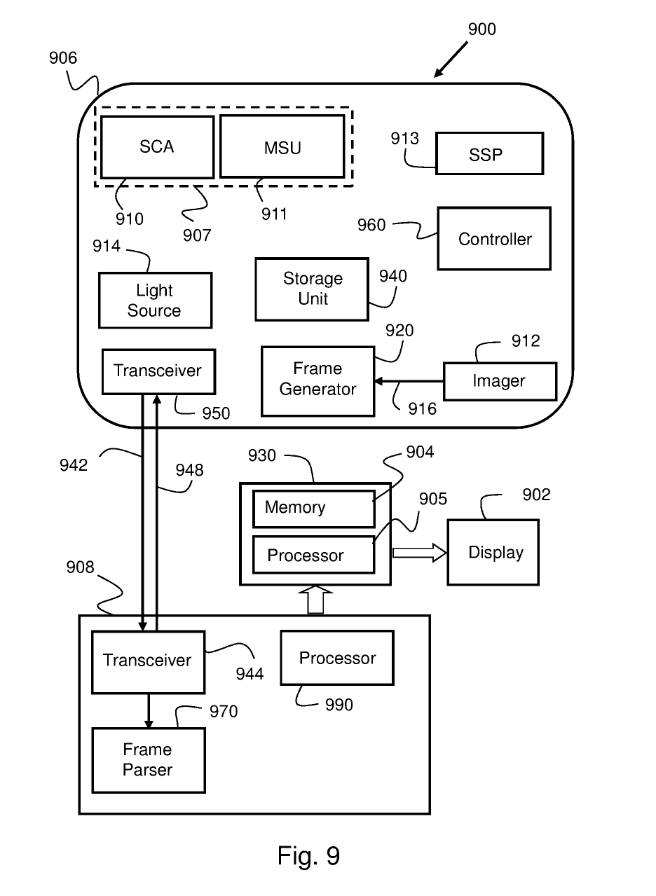

The strength and direction of electrical currents I1_1, I1_2, I2_1 and I2_2 may controllably be manipulated such that magnet 230, hence in-vivo device 240, may be moved upwards/vertically (e.g., in the ‘Y’ direction), or slantingly (at an angle) with respect to axes 250 and 260 (or with respect to the Z direction). (Moving magnet 230 in the Z direction may be obtained or received by generating magnetic field gradient in the Z direction, but this may be implemented using different coils whose normal line coincides with the Z direction, as shown, for example, in FIG. 8B.)

CCA 210 and CCA 220 enable to controllably generate a magnetic field maneuvering pattern (MMP) for maintaining or controlling a current location and/or orientation of magnet 230 in operating region 260, and, if required, for moving magnet 230 in any intended, desired, new or next direction and/or to any intended, desired, new or next orientation in operating region 260. A MMP may generally refer to the strengths and directions of the magnetic field and magnetic gradient at a point or region in an operating space, which is, or that coincides with the current location of the permanent magnet, or with the location of an a device (e.g., swallowable in-vivo device) containing the magnet. Changing an MMP may include or may mean, for example, changing a direction of the magnetic field or magnetic field gradient (or changing both directions) and/or changing their strength/intensity/gradient. A MMP may be controlled by controlling the intensity and direction of magnetic fields 216, 218, 226 and 228 by controlling the magnitude and direction of the respective electrical currents I1_1, I1_2, I2_1 and I2_2.

The magnetic force, F, applied to magnet 230, which depends on the spatial derivative of the magnetic field, may be set to be in a direction of, or at any angle relative to, the magnetic moment, M. Using only one pair of coils (upper coils 212 and 214, or lower coils 222 and 224) may result in a ‘basic’ (Impure′, asymmetric) type of gradient field and a ‘basic’ homogenous (symmetric) magnetic field, BO. However, using the other (opposite) pair of coils enables to cancel the symmetric part of the magnetic field, thus leaving only magnetic force F. Using a coils' system that includes opposite pairs of coils, such as coils pairs 210 and 220, can create magnetic stability (by applying multiple magnetic field gradients that are constant in the space accommodating magnet 230) while superimposing a homogenous magnetic field, BO, that is sufficiently strong to align the magnetic momentum, M, in a desired direction within the space accommodating magnet 230.

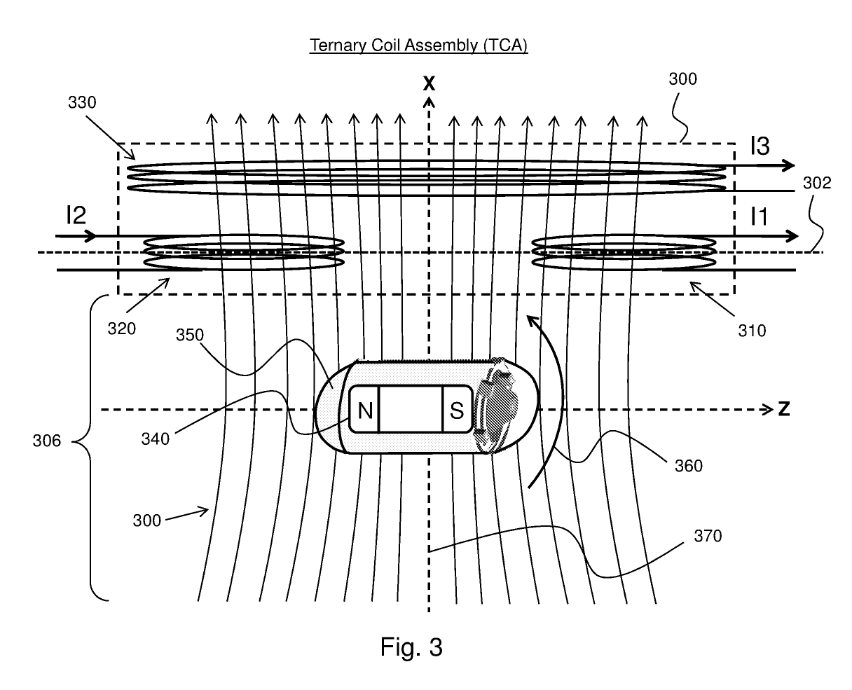

FIG. 3 illustrates a ternary coils assembly 300 according to an example embodiment of the invention. Ternary coils assembly 300 may include a conjugated coils assembly consisting of anterior coil 310 and posterior coil 320, and an ancillary coil 330. Assuming that magnet 340 (and therefore in-vivo device 350) is initially or currently oriented in direction Z, ancillary coil 330 may be used to reorient magnet 340 (and in-vivo device 350), for example, to direction X (370). Reorienting magnet 340 in direction X may be implemented, for example, by energizing/activating only coil 330 (I3>0 Amp; I1=I2=0 Amp) Energizing/activating only ancillary coil 330 may result in magnetic field 300 that is generally vertical (in direction X). Magnetic field 300 may apply a torque on the magnetic moment of magnet 340, causing it to rotate (360) magnet 340 to an angular position compliant with (to align the magnetic moment in) the direction 370 of magnetic field 300.

A counter ternary coils assembly similar to ternary coils assembly 300 may be positioned opposite ternary coils assembly 300 with respect to the circle formed by the TCAs, to avoid the instability problem mentioned above in connection with FIG. 1. (The counter ternary coils assembly is not shown in FIG. 3.) Manipulating electrical currents I1, I2, I3 and the electrical currents flowing in the counter ternary coils assembly enables, for example, manipulating the magnetic field maneuvering pattern (MMP), only this time (e.g., with the ancillary coil 330 and the counter ancillary coil used as well), the ancillary coil may strengthen or weakens a magnetic field generated in a particular direction by the pertinent anterior coils or posterior coils, or by both types of coils (by the conjugated coils assembly/assemblies).

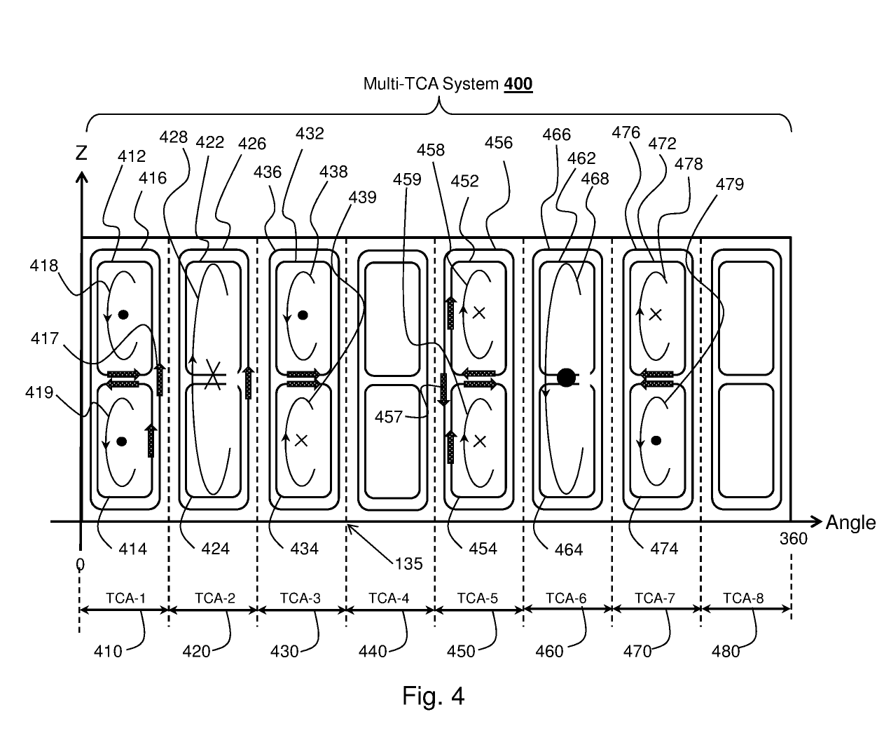

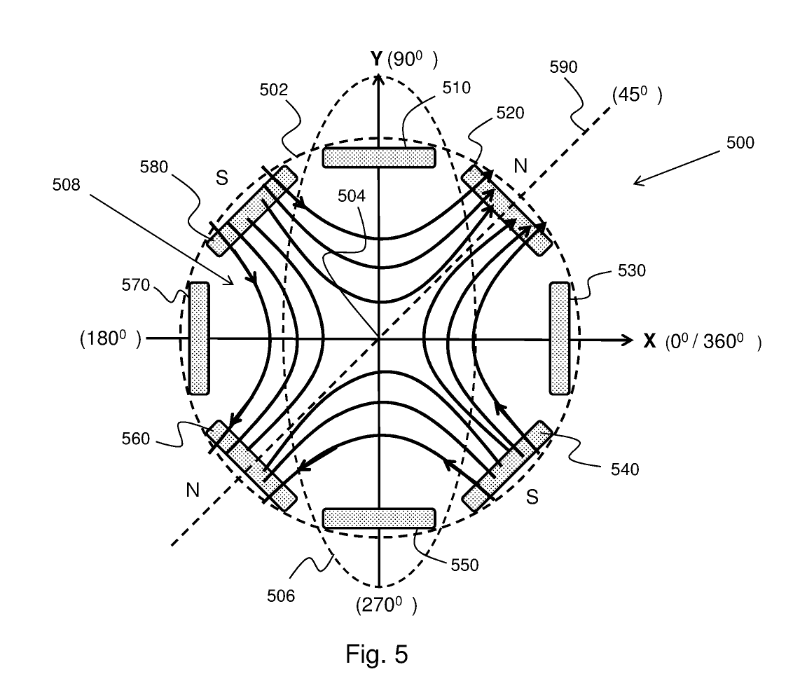

FIG. 4 illustrates a multi-ternary coils assembly 400 (multi-TCA) spread out according to an example embodiment of the invention. By way of example, multi-TCA 400 includes eight ternary coils assemblies (TCAs), designated as TCA-1 (shown at 410), TCA-2 (shown at 420), TCA-3 (shown at 430), TCA-4 (shown at 440), TCA-5 (shown at 450), TCA-6 (shown at 460), TCA-7 (shown at 470) and TCA-8 (shown at 480). (Other numbers of TCAs may be used; e.g., less than eight TCAs; e.g., 2, 3, 4, etc., or e.g., more than eight TCAs; e.g., nine, ten, etc.) TCA-1 through TCA-8, which may form a circle or may be circularly circumscribed, are shown spread out in FIG. 4. (Circularly circumscribed TCAs are shown, for example in FIGS. 5 and 8A.) The vertical axis in FIG. 4 is the Z axis, which may be normal to a plane of the circular multi-TCAs. The horizontal axis is an angle axis corresponding to an angular location on the periphery of the circular multi-TCA whose central axis may coincide with the Z axis. For example, TCA 410 arbitrarily starts at zero degrees; TCA 430 terminates at 135 degrees; and TCA 480 terminates at 360 degrees (that is, TCA 410 and TCA 480 are peripherally adjacent).

Each TCA may include for example three coils: two, conjugated and adjacently positioned side by side, internal coils (one anterior coil and one posterior coil), and a third coil (an ancillary coil) that may encircle the internal coils. For example, TCA 410 may include two internal coils—anterior coil 412 and posterior coil 414—and an ancillary coil 416 that encircles them; TCA 420 may include two internal coils—anterior coil 422 and posterior coil 424—and an ancillary coil 426 that encircles them; TCA 430 may include two internal coils—anterior coil 432 and posterior coil 434—and an ancillary coil 436 that encircles them; TCA 450 may include two internal coils—anterior coil 452 and posterior coil 454—and an ancillary coil 456 that encircles them; TCA 460 may include two internal coils—anterior coil 462 and posterior coil 464—and an ancillary coil 466 that encircles them; TCA 470 may include two internal coils—anterior coil 472 and posterior coil 474—and an ancillary coil 476 that encircles them, and so on. The two internal coils and the ancillary coil of each TCA may electrically be isolated from each other.

Formed as a circle, the TCA assembly including TCA-1 through TCA-8 may function in pairs, meaning that each TCA-i may have a conjugated, oppositely positioned (on the formed circle), TCA-j (j≠i; e.g., j=i+4) with which it may jointly operate. For example, TCA-1 and TCA-5, which is opposite TCA-1 (on the formed circle) and is the conjugated TCA of TCA-1, form a first pair of TCAs; TCA-2 and the opposite TCA (TCA-6), is the conjugated TCA of TCA-2, form a second pair of TCAs; TCA-3 and opposite TCA-7 form a third pair of TCAs, and TCA-4 and opposite TCA-8 form a fourth pair of TCAs. A pair of TCAs, or a combination of pairs of TCAs, or a combination of pair(s) and single TCAs, may jointly generate a wanted MMP to maneuver a permanent magnet, for example a permanent magnet incorporated in an in-vivo device.

TCA-1 through TCA-8 may be operated in various ways (e.g., by the pairs), by individually manipulating the magnitude and direction of the electrical current flowing in each coil of each TCA, to thereby obtain or receive a MMP required to maneuver the in-vivo device to a next location and/or next orientation, or to maintain the in-vivo device's current location and/or current orientation. By way of example, all three currents of TCA-1 (an example TCA) are shown flowing in a counterclockwise (CCW), as shown by arrows 418, 419 and 417 (all three currents of TCA-5, which is TCA-1's conjugated/opposite TCA, are shown flowing in a clockwise, as shown by arrows 458, 459 and 457). Causing all three currents to flow in the same direction enables generating a strong magnetic field in a direction that may be perpendicular to the page's plane. (The magnetic fields resulting from current 418, which flows in anterior coil 412 of TCA-1, and from current 419, which flows in posterior coil 414 of TCA-1, emerge from the page, as shown by the two black dots. The magnetic fields resulting from current 458, which flows in anterior coil 452 of TCA-5, and from current 459, which flows in posterior coil 454 of TCA-5, go through the page, as shown by the two “Xs”.)

According to another example, a magnetic field of a smaller magnitude, and in an opposite direction relative to the direction of the magnetic field generated by TCA-1, may be generated by TCA-2 and its conjugated TCA (e.g., TCA-6), for example, by passing an electrical current only in the pertinent ancillary coil, and in the clockwise (CW) direction. The electrical current flowing in ancillary coil 426 is shown at 428, and the electrical current flowing in ancillary coil 466 is shown at 468. (In this example, the electrical currents flowing in anterior coil 422, posterior coil 424, anterior coil 462 and posterior coil 464, may be zero.

According to another example, a magnetic field having a maneuvering pattern similar to the magnetic field pattern shown in FIG. 2 may be obtained or received by operating the TCA pair including TCA-3 and its conjugated TCA-7 in a similar way as demonstrated, for example, in FIG. 2. For example, such pattern may be produced/obtained by passing a first electrical current in anterior coil 432 in the CCW direction, as shown at 438, a second electrical current in posterior coil 434 in the CW direction, as shown at 439, a third electrical current in anterior coil 472 in the CW direction, as shown at 478, and a fourth electrical current in posterior coil 474 in the CCW direction, as shown at 479. (In this example, the electrical currents in the ancillary coils 436 and 476 may be zero.)

FIG. 5 illustrates multiple circularly positioned ternary coils assemblies (TCAs) 500 according to an example embodiment of the invention. In general, N ternary coil assemblies may be positioned circularly and form a plane (e.g., X-Y plane, as demonstrated by FIG. 5), for example, such that each ternary coil assembly may have a paired, conjugated, ternary coil assembly on the opposite side of the circle formed by the N ternary coils assemblies, and the anterior, posterior and ancillary axes of each ternary coil assembly may be parallel to the plane formed by the multiple TCAs. By way of example, N=8, that is, TCAs 500 may include eight ternary coils assemblies (designated as 510, 520, 530, 540, 550, 560, 570 and 580), which form, or are circularly circumscribed by, a circle 502, or TCAs 500 may form a circle. Circular TCAs 500 may form, or lie on, a plane coinciding with the Cartesian X-Y plane having a normal in the direction of the Z axis. Each of TCAs 510 through 580 includes an anterior coil, a posterior coil and an ancillary coil, as per the description herein of a TCA's structure and functionality. The anterior coil and the posterior coil, though not shown in FIG. 5, are positioned one after another with respect to the Z axis, as implied by the appellation ‘anterior’ and ‘posterior’ and described, for example, in connection with FIG. 2. That is, the N (e.g., N=8) ternary coil assemblies may be positioned circularly and form a plane; e.g., on the X-Y plane, such that all the anterior coils are positioned on one side of the plane (e.g., all anterior coils have positive Z values/coordinates) and all the posterior coils may be positioned on the opposite side of the plane (e.g., all posterior coils have negative Z values/coordinates).

TCAs 510 through 580 may functionally be divided into four pairs of conjugated TCAs, where each pair may include a first TCA and a second TCA that may be positioned opposite the first TCA. The TCAs may equidistantly be distanced from the coordinate's origin 504 and equidistantly spaced apart on circle 502, with the same angle separating any two adjacent TCAs. A pair of TCAs may individually and jointly function or operate in a similar way as TCAs 210 and 220 in FIG. 2.

One example TCA pair (shown at 506) may include two TCAs, designated as 510 and 550, on the Y axis; two TCAs, designated as 520 and 560, in a direction 590 that may be at an angle of 45 degrees with respect to the Y axis; two TCAs, designated as 530 and 570, on the X axis; and two TCAs, designated as 540 and 580, in a direction that may be at an angle of −45 degrees with respect to the X axis.

Any combination of TCAs, including any combination of current magnitudes and current directions, may be used to generate any wanted/desired MMP). As explained herein, a MMP may include a magnetic field whose direction may be in any desired direction, including only the X axis/direction, or in the Y axis/direction, or in the Z axis/direction, or in any intermediate/intervening directions (e.g., at any angle with respect to any axis). An example MMP is shown at 508. Example MMP 508 has magnetic field whose direction(s) is/are in the X-Y plane but not in the Z direction. (Other MMPs may have magnetic field in other directions, including in the Z direction, as explained herein.) MMP 508 may be generated, for example, by using only ancillary coils of TCAs 520, 540, 560 and 580. However, the same, or similar, MMP may be obtained, for example, by jointly operating only anterior coils and their respective posterior coils, or by operating all three coils—ancillary coils, anterior coils and posterior coils, as described above in connection with FIG. 4. Orientation of MMP 508 may controllably be changed to a different orientation by shutting down (switching off the currents passing through) TCAs 520, 540, 560 and 580, and using a different set of TCAs, for example TCAs 510, 530, 550 and 570. Such TCAs swapping may result, for example, in the MMP rotating 45 degrees counterclockwise or 45 degrees clockwise.

The magnetic field generation schemes shown in FIGS. 2 and 5, when used together, provide for enhanced/improved maneuverability, for example, of in-vivo devices, because they jointly enable generating magnetic field in any spatial direction within a three-dimensional operating region, and, in addition, they provide for independent control of the direction of the magnetic field, which aligns or positions a device, and the direction of the magnetic gradient, which is the direction of the force acting on the device.

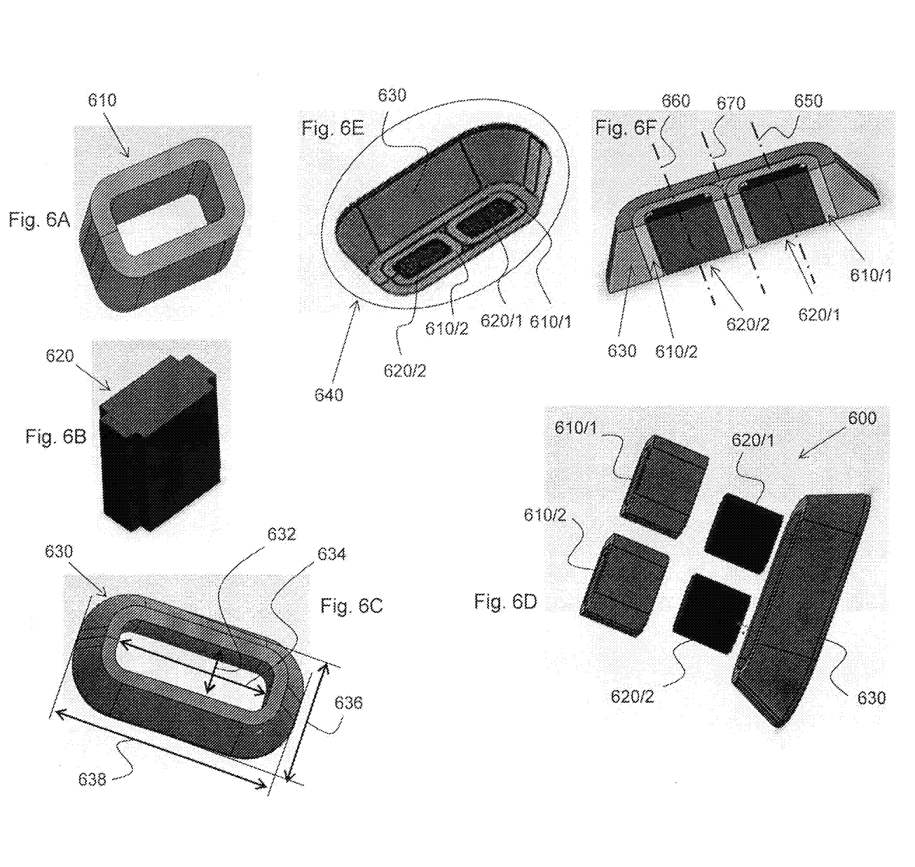

FIGS. 6A-6F depict components of a ternary coil assembly (TCA) and an assembled ternary coil assembly according to an example embodiment of the invention. (Like reference numerals refer to like components.) Referring to FIG. 6A, reference numeral 610 denotes a TCA's internal coil/coil that is referred to herein as an ‘anterior coil’ (or ‘posterior coil’). Referring to FIG. 6B, reference numeral 620 denotes a magnetic core of a TCA's internal coil/coil. Referring to FIG. 6C, reference numeral 630 depicts an object embedding an ancillary coil/coil of the TCA.

FIG. 6D depicts a spread out, or an exploded, TCA 600. TCA 600 may include two internal coils (e.g., anterior coil 610/1 and posterior coil 610/2), two magnetic cores (one for each internal coil; e.g., magnetic core 620/1 for internal coil 610/1 and magnetic core 620/2 for internal coil 610/2). TCA 600 may also include an ancillary coil object/block (e.g., ancillary coil object/block 630) that contains, or embeds, an ancillary coil. Assembling the various components depicted in FIG. 6d results in assembled TCA 640, which is depicted in FIG. 6e. FIG. 6f depicts a cross sectional view of TCA 640 of FIG. 6e. Referring to FIG. 6f, the two internal coils (anterior coil 610/1 and posterior coil 610/2) are shown positioned adjacently, side by side. Reference numeral 650 denotes, for example, an anterior coil axis (it is the axis of anterior coil 610/1), reference numeral 660 denotes, for example, a posterior coil axis (it is the axis of posterior coil 610/2), and reference numeral 670 denotes an ancillary coil axis (it is the axis of ancillary coil 630 object/block). Anterior coil axis 650, posterior coil axis 660, and ancillary coil axis 670 may be mutually parallel, and ancillary coil axis 670 may be in-between anterior axis 650 and posterior axis 660.

Example Mechanical Dimensions and Electrical Parameters of TCA Components

Example specifications of some embodiments are presented below; other specifications may be used with embodiments of the present invention.
1. Ancillary coil: An ancillary coil, which may be identical or similar to ancillary coil 630 of FIG. 6C, may be elliptically shaped (or it may have any other shape; e.g., cylindrical, annular, etc.) and have for example the following dimensions:
   a) Internal width (shown at 632) may be between 100 mm and 300 mm, and the internal length (shown at 634) may be between 300 mm and 900 mm. For example the internal width (632) and length (634) may respectively be 200 mm and 600 mm
   b) External width (shown at 636) may be between 150 mm and 400 mm, and the external length (shown at 638) may be between 400 mm and 1,000 mm. For example the internal width (636) and length (638) may respectively be 300 mm and 700 mm
   c) Foil dimensions (height×thickness):
      (c.1) Height: 50-500 mm (e.g., 200 mm)
      (c.2) Thickness: 0.2 mm-2 mm (e.g., 1 mm)
   d) Electrical current (ampere, A): 100 A-500 A (e.g., 300 A).
      Other parameters, such as overall length of the coil, number of coil turns and inductance may dependent on the implementation. Example data is shown in Table-1 below.

TABLE 1

| | |
|---|---|
| Number of turns | 50 |
| Length [m] | 270 |
| Material of the coil | copper |
| Resistance [mΩ] | 23 |
| Inductance [mH] | 25 |
| Max. Current [A] | 300 |

2. Anterior/posterior coil: An ancillary coil, which may be identical or similar to anterior (and/or posterior) coil 610 of FIG. 6A, may generally be elliptically shaped (but it may have any other shapes; e.g., cylindrical, annular, rectangle, etc.) and may have the following dimensions:
   a) Internal width×length (mm): (50-200)×(100-400) (e.g., 120×220).
   b) External width×length (mm): (100-400)×(150-500) (e.g., 200×300).
   c) Foil dimensions (height×thickness):
      (c.1) Height: 50-500 mm (e.g., 200 mm)
      (c.2) Thickness: 0.2 mm-2 mm (e.g., 1 mm)
   d) Electrical current (ampere, A): 100 A-800 A (e.g., 400 A).
      Other parameters, such as overall length of the coil, number of coil turns and inductance may dependent on the implementation. Example data is shown in Table-2 below.

TABLE 2

| | |
|---|---|
| Number of turns | 40 |
| Length [m] | 75 |
| Material of the coil | copper |
| Resistance [mΩ] | 6.45 |
| Inductance [mH] | 5.4 |
| Max. Current [A] | 400 |

e) Iron Core: the magnetic core (e.g., core 620 of FIG. 6B) may be, or include, a low loss soft iron, and it may have general dimension 215×110×220 (Width/Length/Height) with tolerances:

| | | |
|---|---|---|
| Width: | 215 → | tolerance: 100:400 |
| Length: | 110 → | tolerance: 50:200 |
| Height: | 220 → | tolerance: 50:500 |

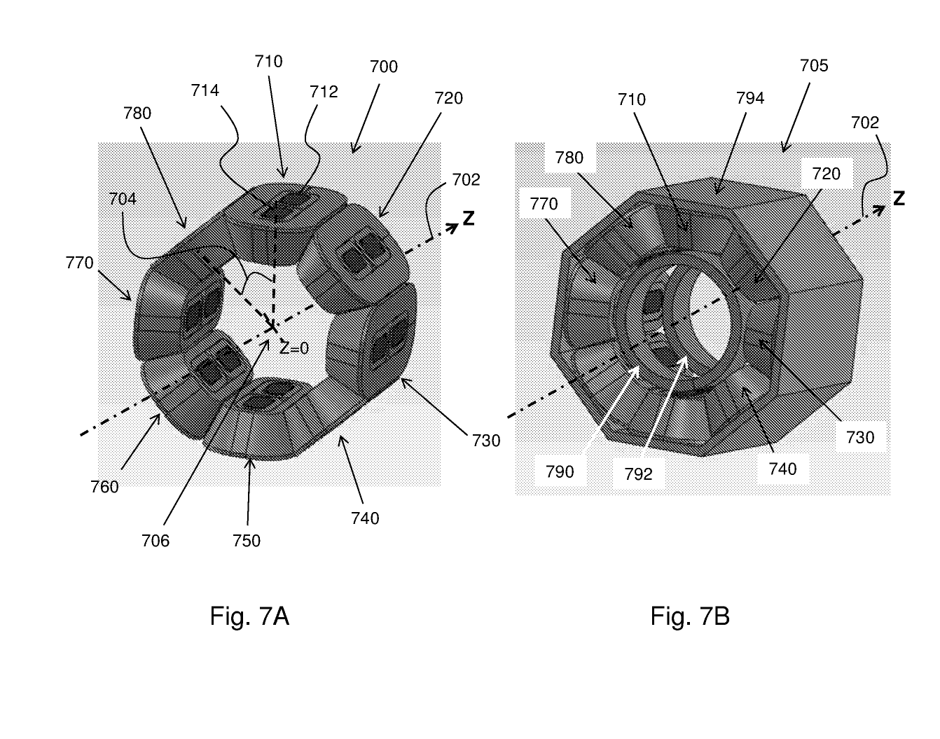

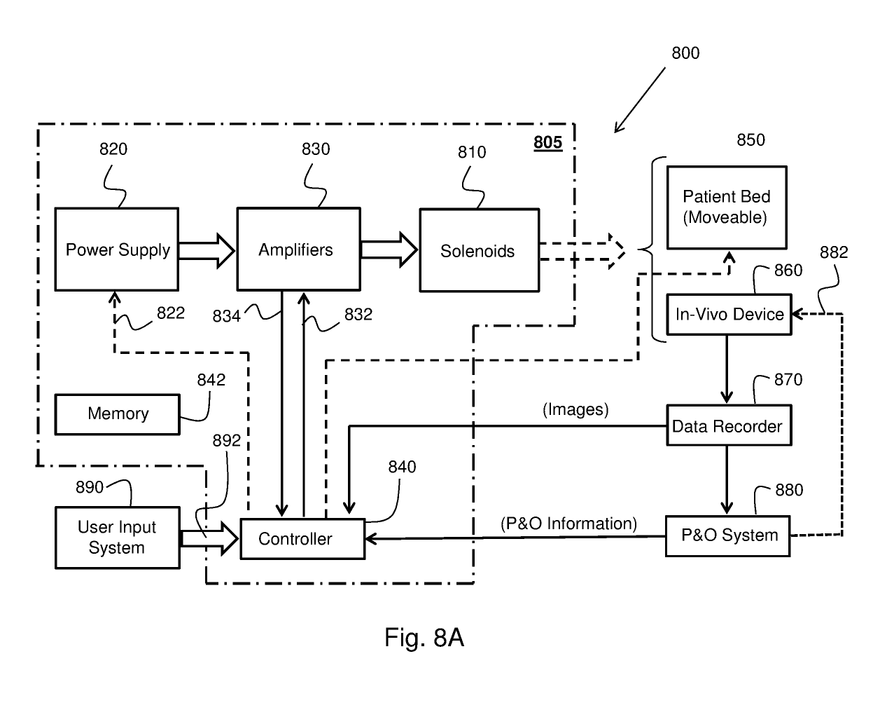

FIG. 7A depicts a circular electromagnets setup 700 according to an example embodiment of the invention. Circular electromagnets setup 700 may include 8, circularly positioned, ternary coil assemblies (TCAs), designated as 710, 720, 730, 740, 750, 760, 770 and 780. Each of TCAs 710 through 780 may resemble, for example, TCA 640 of FIG. 6E. (Circular electromagnets setup 700 may include a number N of TCAs, where N may be an even number; e.g., N may be equal to six, or to eight, or to ten, etc., or N may be odd.) TCAs 710 through 780 may be circular about the Z axis (702), and form a plane, or coincide with a plane defined by the perpendicular X axis and Y axis. (Axes X and Y are not shown in FIG. 7A.) TCAs 710 through 780 may form a circle. Assuming that the X-Y plane is located at coordinate Z=0, the Z value of every anterior coil (e.g., anterior coil 712) may be positive (+Z), whereas the Z value of every posterior coil (e.g., posterior coil 714) may be negative (−Z). That is, the N ternary coil assemblies may be positioned circularly and form a plane; e.g., on the X-Y plane, such that all the anterior coils are positioned on one side of the plane (e.g., on the positive side of the Z axis) and all the posterior coils are positioned on the opposite side of the plane (e.g., on the negative side of the Z axis). Each anterior coil has an anterior axis; each posterior coil has a posterior axis, and each ancillary coil has an ancillary axis. (The three axes may mutually be parallel.) The N ternary coil assemblies may be positioned circularly and form a plane such that the anterior, posterior and ancillary axes of each ternary coil assembly are substantially parallel to the plane, and such that each ternary coil assembly has a paired, conjugated, ternary coil assembly on the opposite side of the circular N ternary coils assemblies,

In general, the angular spacing (a) between each two adjacent TCAs may be equal to 360/N, where N is the number of TCAs in the circular electromagnets setup. Referring to FIG. 7A, as an example circular electromagnets setup, N is equal to eight. Therefore, the angular spacing, a, between each two adjacent TCAs of TCAa 710 through 780 is α=360/8=45 degrees, as shown at 704, which is the angular spacing between TCA 710 and TCA 780. (The angular spacing between TCAs is in the X-Y plane.)

Electromagnets setup 700 may enable controllably generating an MMP for maintaining or controlling a current location and/or an orientation of a magnet, or of an in-vivo device containing a magnet or attached to a magnet, in an operating region 706. If required, a corresponding MMP may be applied to the magnet in order to move the magnet, or the device containing or attached to the magnet, in any intended (e.g., by a user/operator, or by a system outputting a signal embodying the intention), desired, new or next direction and/or to any intended, desired, new or next orientation within operating region 706.

FIG. 7B depicts a maneuvering coils setup or magnetic system 705 that includes the circular electromagnets setup 700 of FIG. 7A and ‘Z’ coils according to an example embodiment of the invention. A ‘Z-coil’ is a coil lying on or coinciding with the X-Y plane which is perpendicular to the Z axis (702). (A Z-coil may form a plane having a normal in the Z direction.) A Z-coil may generate a magnetic field, and a magnetic field gradient, in the Z direction (e.g., in the +Z direction or in the −Z direction). FIG. 7B depicts two Z-coils, designated as 790 and 792. Additional Z-coils may be interposed in-between Z-coils 790 and 792 to enhance the magnetic field in the Z direction, for example to make the magnetic field in the Z more uniform and, if required, stronger. The TCAs 710 through 780 and Z-coils 790 and 792 are shown encapsulated by a housing, or shell, that, for example, may be octagon, as shown at 794.

FIG. 8A is a block diagram of a magnetic maneuvering system 800 according to an example embodiment of the invention. Maneuvering system 800 may include a magnetic maneuvering control system 805, a patient bed 850 on which a subject may lie, data recorder/receiver 870, and position and orientation (“P&O”) system 880. An in-vivo device 860 may be swallowed by the subject before, or while, the subject lies on bed 850. Bed 850 may be moveable.

Magnetic maneuvering control system 805 may include a maneuvering coils system 810, which may be identical or similar to maneuvering coils system, or magnetic system, 705 of FIG. 7B, a power supply 820 that may be configured to provide electrical currents to the TCAs of maneuvering coils system 810, amplifiers 830 (e.g., an amplifier per coil) for controllably and individually adjusting the electrical current flowing through each coil/coil of maneuvering coils system 810, a controller 840 for controlling a MMP, for example, by changing the electrical state of the amplifiers by individually controlling an electrical parameter (e.g., amplification/gain, conduction, etc.) of each amplifier; and a memory 842. Memory 842 may store an instruction code for controller 840 to execute, P&O information/data, user input data, etc.

In operation, a subject having ingested in-vivo device 860 lies on bed 850. In-vivo device 860 may wirelessly transmit image data pertaining to images acquired in the subject's GI system, and possible data of other type(s), to data recorder/receiver 870. (Recorder/receiver 870 may be incorporated into or attached to a gantry containing coils 810.) Recorder/receiver 870 may transfer image data, and possibly other types of data, to controller 840. Controller 840 may display transferred images to a display device, and use them, for example, to navigate or maneuver in-vivo device 860 in the subject's GI system.

P&O system 880 may transmit localization signals (882) in proximity to the subject's GI system, and a localization signal sensor (e.g., sensing coil(s)) residing in in-vivo device 860 may sense the localization signals and transmit raw, pre-processed or fully processed P&O data, which are related to the in-vivo device's current P&O, to data recorder 870. Data recorder 870 may transfer the P&O data pertaining to the current device's P&O to P&O system 880, and P&O system 880 may process the P&O data and transfer P&O information to controller 840.

A user (e.g., a physician) may use an input system 890 to transfer P&O data to controller 840 regarding a desired P&O or a next or intended P&O of in-vivo device 860. Controller 840 may analyze the next/intended P&O vis-a-vis the current P&O information and, based on the analysis, controller 840 may determine the magnitude and/or direction of the electrical current that should be provided to each one of coils (electromagnets) 810. Controller 840 may, then, output/transfer (832) a corresponding signal, or signals, to amplifiers 830 to accordingly adjust an electrical parameter of one or more of amplifiers 830. The adjusted electrical currents, then, may be fed or provided to coils 810 to maneuver in-vivo device 860 to the intended/new P&O. Controller 840 may monitor (834), for example it may get feedback regarding, the state of amplifiers 830, and use the feedback information, in conjunction with the P&O data/information, to control the state of amplifiers 830. Controller 840 may also control (822) power supply 820, for example, to change the electrical currents' dynamic range. That is, controller 840 may control the various electrical currents coarsely by controlling the electrical state of power supply 820, and fine-tune the electrical currents by using amplifiers 830.

Controller 840 may be configured to operate, or by operating, N ternary coil assemblies to generate a MMP at a location of in-vivo device 860 within an operating region, such that the magnetic field subject of the MMP may be in a first direction (to orient the device in that direction), and a magnetic gradient of the magnetic field may be in a second direction (to apply movement force on the device in that, second, direction). The direction of the magnetic field gradient (the second direction) may be, for example, parallel to the first direction (the direction of the magnetic field), or the second direction may be at angle alpha (a) with respect to the first direction. For example, alpha (a) (the angle between the first and second directions) may have a value within the range of 0 degrees to 90 degrees (e.g., alpha may be an acute angle or an obtuse angle). The direction of the magnetic field (the first direction), or magnetic field gradient, or both, may be parallel to, or perpendicular to, or at an angle beta ((3) with respect to the normal of the plane of, or formed by, the N ternary coil assemblies.

Controller 840 may also be configured to selectively activate, for one or more ternary coil assemblies, a coil selected from the group consisting of: anterior coil, posterior coil and ancillary coil, in order to generate a wanted or required MMP. That is, not all coils (anterior, posterior and ancillary) of a TCA have to be activated/used; only one (e.g., ancillary coil) or two (anterior and posterior coils) may be activated/used. The number and circular position of TCA coils used, and the way they are used—in terms of current magnitude and direction—may depend on the required MMP. Controller 840 may also be configured to selectively activate a particular pair of ternary coil assemblies in order to generate (i) a magnetic field in a first direction, and (ii) a magnetic field gradient in a second direction similar to or different from the first direction; e.g., at angle alpha (a) with respect to the first direction. Control 840 may control the directions of the magnetic field and magnetic field gradient independently.

Controller 840 may simultaneously, or substantially at the same time, generate a homogeneous magnetic field to align or position an in-vivo device in a first direction, and a magnetic field gradient to exert/apply force to the in-vivo device in a second direction, to thereby manipulate the location and/or orientation of the in-vivo device.

Controller 840 may receive (e.g., from P&O system 880) a P&O data representative of a current P&O of in-vivo device 860, for example, in the GI system of a subject positioned in the circular N ternary coil assemblies (e.g., ternary coil assemblies setup 705), and it may obtain (892) data representative of an intended P&O of the in-vivo device in the GI system. Controller 840 may, then, generate a control signal (832) for the N ternary coil assemblies (e.g., a control signal for each assembly) based on the P&O data and the intended P&O. an example signal may include an instruction to change an electrical parameter of the amplifiers; e.g., an electrical parameter of an amplifier for each TCA. The gain of each of amplifiers 830 may be an example electrical parameter. The control signal may be signal that is provided to an input terminal of an amplifier.

FIG. 8B is a partial block diagram of a magnetic maneuvering control system 805′ according to an example embodiment of the invention. Magnetic maneuvering control system 805′ may include N TCAs, designated as TCA-1 (shown at 812), TCA-2 (shown at 814), . . . , TCA-N. (All the TCAs are shown at 810′.) Each TCA may include three coils—an anterior coil, a posterior coil and an ancillary coil. For example, TCA-1 may include a first coil ‘Coil 1_1’ (“Solenoid 1_1”), which may be an anterior coil, a second ‘Coil 1_2’ (“Solenoid 1_2”), which may be a posterior coil, and a third ‘Coil 1_3’ (“Solenoid 1_3”), which may be an ancillary coil. Each TCA may function in the way described herein.

Magnetic maneuvering control system 805 may also include 3×N amplifiers, as shown at 830′, for example one amplifier per one coil. For example, amplifier ‘Amplifier 1_1’ may drive Solenoid 1_1, amplifier ‘Amplifier 1_2’ may drive Solenoid 1_2, and so on. (Other amplifier configurations may be used.)

An in-vivo device (e.g., in-vivo device 860) may be maneuvered from one position to another by using more than one current solution. (′Current solution′ refers to a set of coil currents that jointly generate the MMP required to maneuver an in-vivo device to a next P&O.) It has been contemplated by the inventors that there can be more than one current solution to a particular MMP; namely, the same, or similar, MMP may be generated by using different sets of coils and coil currents. However, the overall electrical power consumed from the system's power supply (e.g., power supply 820) in order to drive the system's coils may vary between sets of coils and currents. That is, a particular set of coil currents may be more economical than other sets, which calls for optimization process with respect to which set of coil currents better suits a particular maneuvering requirement or MMP in terms of electrical power. Controller 840 may, in selecting coils and coils' currents, execute an optimization procedure/program to determine which set of coils and coil currents are optimal (in terms of power consumption) for any maneuvering requirement (e.g., for generating a MMP for maneuvering a device from one point to another in a three-dimensional operating region). Given a particular maneuvering requirement or MMP, the optimization process may include a step of calculating one or more sets of coil currents, and another step of selecting for operation/usage, or applying, the set of coil currents that results in the minimum electrical power consumption. Below are some non-limiting examples of coils and coil currents selections for generating example magnetic field maneuvering patterns according to embodiments of the invention.

### Example-1

Table-3 below pertains to a case where it is required to levitate an in-vivo device, for example, in the stomach while a field of view (FOV) of an imager of the in-vivo device is in the +Z direction. (The in-vivo device ‘looks’ in the positive direction of the Z axis and ‘floats’, or levitates, with respect to the Y axis, which coincides with the gravity force's direction.) While the direction of the magnetic field (at the in-vivo device), in this example, coincides with the Z direction in order to align or position the in-vivo device imager in the Z direction), the magnetic gradient is, in this example, in the Y direction to generate a force that counteracts the gravity force. The magnetic field strength is, in this example, 800 gauss/meter. Table-3 specifies the electrical current in each coil of each TCA. (Eight TCAs are assumed.)

TABLE 3

| Coil (A) | Coil Name/Position | Coil Type |
|---|---|---|
| 52.8 | X | Ancillary Coil - 1 |
| 14.9 | X + 45 | Ancillary Coil - 2 |
| −29.6 | X + 90 | Ancillary Coil - 3 |
| 11 | X + 135 | Ancillary Coil - 4 |
| −58.2 | X + 180 | Ancillary Coil - 5 |
| −4.59 | X + 225 | Ancillary Coil - 6 |
| 33.3 | X + 270 | Ancillary Coil - 7 |
| −6.21 | X + 315 | Ancillary Coil - 8 |
| 10.75 | 180 (−Z) | Posterior coil |
| 34.8 | 180 (+Z) | Anterior coil |
| 348.5 | 225 (−Z) | Posterior coil |
| −319 | 225 (+Z) | Anterior coil |
| 350 | 270 (−Z) | Posterior coil |
| −344 | 270 (+Z) | Anterior coil |
| 299.5 | 315 (−Z) | Posterior coil |
| −239.5 | 315 (+Z) | Anterior coil |
| 201.5 | 000 (−Z) | Posterior coil |
| 155.5 | 000 (+Z) | Anterior coil |
| −42.45 | 45 (−Z) | Posterior coil |
| 323.5 | 45 (+Z) | Anterior coil |
| −290 | 90 (−Z) | Posterior coil |
| 350 | 90 (+Z) | Anterior coil |
| −215 | 135 (−Z) | Posterior coil |
| 313 | 135 (+Z) | Anterior coil |
| −14.7 | Z1 (+Z) | First Z-coil |
| 100 | Z1 (−Z) | Second Z-coil |
| −57.7 | Z2 (+Z) | Third Z-coil |
| 20.7 | Z2 (−Z) | Fourth Z-coil |

Legend: (only representative examples are described below. The legend is applicable also to the other tables below. ‘X’, ‘Y’ and ‘Z’ are Cartesian axes of a gantry housing or enclosing the maneuvering coils, where ‘Z’ coincides with a normal of a plane of the maneuvering coils.)
1. Coil name and position X (Ancillary Coil)—this is an ancillary coil positioned on the X axis, at X=0 degrees (e.g., as in TCA 530 in FIG. 5).
2. Coil name and position X+45 (Ancillary Coil)—this is an ancillary coil positioned between the X axis and the Y axis; i.e., at 45 degrees with respect to the X axis (e.g., as in TCA 520 in FIG. 5).
3. Coil name and position X+90 (Ancillary Coil)—this is an ancillary coil positioned on the Y axis; i.e., at 90 degrees with respect to the X axis (e.g., as in TCA 510 in FIG. 5).
4. Coil name and position X+135 (Ancillary Coil)—this is an ancillary coil positioned between the X axis and the Y axis; i.e., at 135 degrees with respect to the X axis (e.g., as in TCA 580 in FIG. 5), etc.
5. Coil name and position 180(−Z) (Posterior coil)—this is a posterior coil positioned on the (−X) axis, i.e., at 180 degrees with respect to the X axis (e.g., as in TCA 570 in FIG. 5), and located on the negative side of the Z axis (have negative Z value).
6. Coil name and position 180(+Z) (Anterior coil)—this is an anterior coil positioned on the (−X) axis, i.e., at 180 degrees with respect to the X axis (e.g., as in TCA 570 in FIG. 5), and located on the positive side of the Z axis (have positive Z value). The coils at positions 180(−Z) and 180(+Z) may make up or form conjugated coils that may form a TCA (e.g., TCA 570) with the ancillary coil at position X+180.
7. Coil name and position 315(−Z) (Posterior coil)—this is a posterior coil positioned between the X axis and the (−Y) axis, i.e., at 135 degrees with respect to the X axis (e.g., as in TCA 540 in FIG. 5), and located on the negative side of the Z axis (have negative Z value).
8. Coil name and position 315(+Z) (Anterior coil)—this is an anterior coil positioned between the X axis and the (−Y) axis, i.e., at 135 degrees with respect to the X axis (e.g., as in TCA 540 in FIG. 5), and located on the positive side of the Z axis (have positive Z value). The coils at positions 315(−Z) and 315(+Z) may make up or form conjugated coils that may form a TCA (e.g., TCA 540) with the ancillary coil at position X+315.
9. Coils Z1(+Z) and Z2(+Z) are, respectively, a first coil and a second coil whose normal lines coincide with the Z axis/direction. One of the two coils/coils may be positioned in front of the other soil with respect to the positive direction of the Z axis. For example, coil Z1(+Z) may be positioned in front of coil/coil Z2(+Z). The same may apply to soils Z1(−Z) and Z2(−Z), but in the opposite/negative direction of the Z axis.

### Example-2

Table-4 below shows a case where it is required to levitate an in-vivo device in the stomach while the imager's FOV is now in the +X direction. (The in-vivo device ‘looks’ in the positive direction of the X axis and ‘floats’, or levitates, with respect to the Y axis.) While the direction of the magnetic field (at the in-vivo device), in this example, coincides with the +X direction in order to align or position the in-vivo device imager in the +X direction), the magnetic gradient is, in this example, in the Y direction to generate a force that counteracts the gravity force. The magnetic field strength is, in this example, 800 gauss/meter. Table-4 specifies the electrical current in each coil of each TCA. (Eight TCAs are assumed.)

TABLE 4

| Coil (A) | Coil Name/Position | Coil Type |
|---|---|---|
| 4.07 | X | Ancillary Coil -1 |
| 84.4 | X + 45 | Ancillary Coil -2 |
| 53 | X + 90 | Ancillary Coil -3 |
| −84.1 | X + 135 | Ancillary Coil -4 |
| −154 | X + 180 | Ancillary Coil -5 |
| 51.1 | X + 225 | Ancillary Coil -6 |
| 31.8 | X + 270 | Ancillary Coil -7 |
| −48.9 | X + 315 | Ancillary Coil -8 |
| 93 | 180 (−Z) | Posterior coil |
| 88.5 | 180 (+Z) | Anterior coil |
| 18.75 | 225 (−Z) | Posterior coil |
| 16.55 | 225 (+Z) | Anterior coil |
| 4.31 | 270 (−Z) | Posterior coil |
| 1.635 | 270 (+Z) | Anterior coil |
| −21.55 | 315 (−Z) | Posterior coil |
| −20.65 | 315 (+Z) | Anterior coil |
| 36.6 | 000 (−Z) | Posterior coil |
| 37.3 | 000 (+Z) | Anterior coil |
| −2.1 | 45 (−Z) | Posterior coil |
| −1.185 | 45 (+Z) | Anterior coil |
| −111.5 | 90 (−Z) | Posterior coil |
| −117 | 90 (+Z) | Anterior coil |
| 45.95 | 135 (−Z) | Posterior coil |
| 89.1 | 135 (+Z) | Anterior coil |
| −39.3 | Z1 (+Z) | First Z-coil |
| −46.6 | Z1 (−Z) | Second Z-coil |
| −10.8 | Z2 (+Z) | Third Z-coil |
| −3.74 | Z2 (−Z) | Fourth Z-coil |

### Example-3

Table-5 below shows a case where it is required to levitate an in-vivo device in the stomach while the imager's FOV is in now the +Y direction. (The in-vivo device ‘looks’ in (oriented to) the positive direction of the Y axis and ‘floats’, or levitates, with respect to the Y axis.) Since in this example the in-vivo device has to float and have its FOV directed to the Y direction, both the magnetic field (at the in-vivo device) and the magnetic field gradient coincide with the +Y direction (The magnetic field aligns the in-vivo device imager in the +Y direction and the magnetic gradient generates a force counteracting the gravity force.) The magnetic field strength is, in this example, 800 gauss/meter. (Eight TCAs are assumed.)

TABLE 5

| Coil (A) | Coil Name/Position | Coil Type |
|---|---|---|
| 59.1 | X | Ancillary Coil - 1 |
| −80.6 | X + 45 | Ancillary Coil - 2 |
| −125 | X + 90 | Ancillary Coil - 3 |
| −51.9 | X + 135 | Ancillary Coil - 4 |
| 43.5 | X + 180 | Ancillary Coil - 5 |
| −27.2 | X + 225 | Ancillary Coil - 6 |
| 26.6 | X + 270 | Ancillary Coil - 7 |
| 21.9 | X + 315 | Ancillary Coil - 8 |
| 35.1 | 180 (−Z) | Posterior coil |
| 32.95 | 180 (+Z) | Anterior coil |
| 21.4 | 225 (−Z) | Posterior coil |
| 19.4 | 225 (+Z) | Anterior coil |
| −43.35 | 270 (−Z) | Posterior coil |
| −41.7 | 270 (+Z) | Anterior coil |
| −47.85 | 315 (−Z) | Posterior coil |
| −49.9 | 315 (+Z) | Anterior coil |
| 40.85 | 000 (−Z) | Posterior coil |
| 38.7 | 000 (+Z) | Anterior coil |
| 46.05 | 45 (−Z) | Posterior coil |
| 44.6 | 45 (+Z) | Anterior coil |
| −17.3 | 90 (−Z) | Posterior coil |
| −22.75 | 90 (+Z) | Anterior coil |
| −31.65 | 135 (−Z) | Posterior coil |
| −33.1 | 135 (+Z) | Anterior coil |
| 42.3 | Z1 (+Z) | First Z-coil |
| 13.9 | Z1 (−Z) | Second Z-coil |
| −57.4 | Z2 (+Z) | Third Z-coil |
| −7.9 | Z2 (−Z) | Fourth Z-coil |

### Example-4

Table-6 below refers to a case where it is required to move an in-vivo device, for example, in the small intestine, for example in the Z direction, while the imager's FOV is oriented to the same direction. Since, in this example, the in-vivo device is suspended by the small intestine, there is no need to generate a magnetic gradient in the Y direction to counter gravity force. Therefore, only force in the movement direction (in the Z direction) is required. In addition, since the in-vivo device's FOV is aligned with the movement direction, the magnetic field, which aligns the device's FOV, and the magnetic gradient that causes the force, are in the same direction (in this example in the Z direction). The magnetic field strength is 8,000 gauss/meter. Table-6 specifies the electrical current in each coil of each TCA. (Eight TCAs are assumed.)

TABLE 6

| Coil (A) | Coil Name/Position | Coil Type |
|---|---|---|
| 232 | X | Ancillary Coil - 1 |
| 297 | X + 45 | Ancillary Coil - 2 |
| 296 | X + 90 | Ancillary Coil - 3 |
| 194 | X + 135 | Ancillary Coil - 4 |
| 292 | X + 180 | Ancillary Coil - 5 |
| 288 | X + 225 | Ancillary Coil - 6 |
| 272 | X + 270 | Ancillary Coil - 7 |
| 231 | X + 315 | Ancillary Coil - 8 |
| 342 | 180 (−Z) | Posterior coil |
| 346.5 | 180 (+Z) | Anterior coil |
| 335.5 | 225 (−Z) | Posterior coil |
| 350 | 225 (+Z) | Anterior coil |
| 335.5 | 270 (−Z) | Posterior coil |
| 339.5 | 270 (+Z) | Anterior coil |
| 338.5 | 315 (−Z) | Posterior coil |
| 338.5 | 315 (+Z) | Anterior coil |
| 342.5 | 000 (−Z) | Posterior coil |
| 342 | 000 (+Z) | Anterior coil |
| 343.5 | 45 (−Z) | Posterior coil |
| 340.5 | 45 (+Z) | Anterior coil |
| 344 | 90 (−Z) | Posterior coil |
| 338 | 90 (+Z) | Anterior coil |
| 343 | 135 (−Z) | Posterior coil |
| 341 | 135 (+Z) | Anterior coil |
| −158 | Z1 (+Z) | First Z-coil |
| −141 | Z1 (−Z) | Second Z-coil |
| −167 | Z2 (+Z) | Third Z-coil |
| −140 | Z2 (−Z) | Fourth Z-coil |

### Example-5

Table-7 below shows a case where it is required to move an in-vivo device, for example, in the small intestine, for example in the Y direction, while the imager's FOV is oriented to the same direction (to the Y direction).

Since, in this example, the in-vivo device is suspended by the small intestine, there is no need to generate a force to counter gravity force. Therefore, only movement force in the movement direction (in the Y direction) is required. In addition, since the in-vivo device's FOV is to be aligned with the movement direction (Y), the magnetic field, which aligns/orients the device's FOV, and the magnetic gradient, which applies the movement force, are in the same direction (in this example in the Y direction). The magnetic field strength is, in this example, 8,000 gauss/meter. Table-7 specifies the electrical current in each coil of each TCA. (Eight TCAs are assumed.)

TABLE 7

| Coil (A) | Coil Name/Position | Coil Type |
|---|---|---|
| 300 | X | Ancillary Coil - 1 |
| −300 | X + 45 | Ancillary Coil - 2 |
| −300 | X + 90 | Ancillary Coil - 3 |
| −300 | X + 135 | Ancillary Coil - 4 |
| 300 | X + 180 | Ancillary Coil - 5 |
| −217 | X + 225 | Ancillary Coil - 6 |
| −300 | X + 270 | Ancillary Coil - 7 |
| −121 | X + 315 | Ancillary Coil - 8 |
| 350 | 180 (−Z) | Posterior coil |
| 350 | 180 (+Z) | Anterior coil |
| −350 | 225 (−Z) | Posterior coil |
| −350 | 225 (+Z) | Anterior coil |
| −350 | 270 (−Z) | Posterior coil |
| −350 | 270 (+Z) | Anterior coil |
| −350 | 315 (−Z) | Posterior coil |
| −350 | 315 (+Z) | Anterior coil |
| 350 | 000 (−Z) | Posterior coil |
| 350 | 000 (+Z) | Anterior coil |
| −35.35 | 45 (−Z) | Posterior coil |
| −45.05 | 45 (+Z) | Anterior coil |
| −350 | 90 (−Z) | Posterior coil |
| −350 | 90 (+Z) | Anterior coil |
| −156.5 | 135 (−Z) | Posterior coil |
| −179 | 135 (+Z) | Anterior coil |
| 168 | Z1 (+Z) | First Z-coil |
| 168 | Z1 (−Z) | Second Z-coil |
| 168 | Z2 (+Z) | Third Z-coil |
| 168 | Z2 (−Z) | Fourth Z-coil |

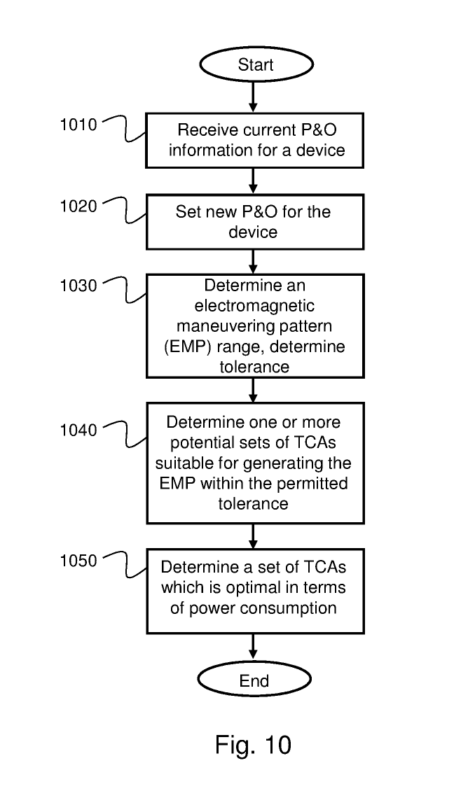

FIG. 9 shows an in-vivo imaging system 900 according to an example embodiment. While FIG. 9 references an in-vivo device (in-vivo device 906) that transmits data frames that may be related to or include any type of sensory data (e.g., pH data), FIG. 9 shows in-vivo device 906 with an imager as an example sensor, in which case in-vivo device 906 may be referred to as an “in-vivo imaging device” or an “in-vivo imager”, and (data) frames transmitted by or from in-vivo device 906 may be referred to as “image frames” (although image frames may include also other types of data, including localization data and/or other types of sensory data. In-vivo imaging system 900 may include in-vivo device 906, data recorder 908, a user workstation 930, which may be, for example, a workstation or personal computer, and a display device 902 for displaying, for example, images and/or a video clip or moving image stream, and for displaying location and/or orientation of the in-vivo device, etc.

An in-vivo imaging device may have or include one or more imagers. By way of example, in-vivo device 906 includes one imager (e.g., imager 912) (numbers of imagers other than one or two may be used). In-vivo device 906 may also include a light/illumination source 914 for illuminating a GI section/site/organ to be imaged, a frame generator 920 for producing an image frame for each captured image, a controller 960, which may execute steps or procedure(s) executed by controller 840, a storage unit 940 for storing data, a transmitter or transceiver 950 for transmitting (942) image frames and, optionally, for receiving (948) data and/or commands from data recorder 908, and an electrical power source for powering these components and circuits. A power source powering in-vivo device 906 may include a charge storing device (e.g., one or more batteries, which may be rechargeable or not) with an electrical circuit that jointly effect transfer of electrical power from an external power source to the in-vivo device through electromagnetic induction.

In-vivo device 906 may include a location and steering unit (LSU) 907. LSU 907 may include a sensing coil assembly (SCA) 910 for sensing localization signals generated, for example, by an external localization system (not shown). SCA 910 may include k electromagnetic sensing coils for sensing, through electromagnetic induction, electromagnetic localization fields/signals, where n is an integer equal to or greater than 1 (e.g., k=2 sensing coils, or k=3 sensing coils) that may be, for example, mutually perpendicular. Each electromagnetic sensing coil may be used to sense an electromagnetic field in a different direction/orientation. For example, one coil may be used to sense an electromagnetic field in the ‘X’ direction or in the Y-Z plane; another coil may be used to sense an electromagnetic field in the ‘Y’ direction or in the X-Z plane, etc. Each localization signal generated by the external localization system may induce an electromagnetic field (EMF) signal on one or more of the k electromagnetic sensing coils of SCA 910, and the current location, and optionally the current orientation, of in-vivo device 906 may be determined based on the EMF signal(s) sensed by (induced in) the sensing coils of SCA 910.

In-vivo device 906 may also include a magnetic steering unit (MSU) 911 to facilitate magnetic maneuvering of in-vivo device 206, for example through interaction with magnetic fields which may be generated by a maneuvering system identical or similar to the magnetic maneuvering system of FIGS. 8A-8B. MSU 911 may include, for example, one or more permanent magnets that may interact with the magnetic field(s) generated, for example, by the magnetic maneuvering system 805 of FIG. 8A, or by a similar system. The permanent magnet(s), in conjunction with the magnetic field(s), may produce magnetic forces and/or torques to steer in-vivo device 906 in a desired direction or to orient or position it in a desired orientation/direction. Controller 960 may transfer, through transceiver 950, data representative of the sensed localization signal. In-vivo device 906 may also include sensing signal processor (SSP) 913 for measuring the EMF signals induced in SCA 910.

Data representing, or derived from, the EMF signals induced in SCA 910 may be transmitted 942, for example, to data recorder 908 by embedding the data in image frames and/or by using frames that may be dedicated to transfer of such data. Frames generator 920 may receive (916) image data that represents a captured image, and produce a corresponding image frame (or “frame” for short) that contains image data. A frame typically includes a header field that contains information and/or metadata related to the frame itself (e.g., information identifying the frame, the serial number of the frame, the frame's generation time, the bit-wise length of the frame, etc.), and a payload field. The payload field may include an uncompressed version of the image data and/or a compressed version thereof, and a decimated image. The payload may also include additional information related to or representative of, for example, values read out from SCA 910.

Controller 960 may operate, among other things, illumination/light source 914 to illuminate areas traversed by in-vivo device 906, and schedule the images capturing times accordingly. Controller 960 may use a timer to time the operation of illumination source 914 to illuminate k times per second (e.g., k=4) to enable capturing k images per second, and the operation of transceiver 950 to concurrently transmit frames at the same rate or at a different rate. Controller 960 may temporarily store captured images and related image frames in data storage unit 940. Controller 960 may also perform various calculations and store interim calculation results in data storage unit 940. Controller 960 may also use the timer to read the EMF output of SCA 910 at an allocated sensing window(s) from which the position and/or orientation of in-vivo device 906 may be calculated or deduced (e.g., by controller 960 or by an external system; e.g., data recorder 908).

Controller 960 may also use the timer to time the writing (e.g., adding, appending, or otherwise embedding) of localization data (e.g., the sensing coils readout or a manipulated version thereof) into the corresponding frame. After frames generator 920 produces a frame for a captured image and embeds localization data in the frame, controller 960 may use transceiver 950 to wirelessly transfer 942 the frame to data recorder 908. Controller 960, by executing software or instructions, may carry out steps which are performed by any one of SSP 913 and frame generator 920, and other functions in in-vivo device 906, and thus may function as these units. For example, controller 960 may thus be configured to carry out embodiments of the present invention. Each of sensing signal processor (SSP) 913 and frame generator 920, and other functions may be implemented as a dedicated hardware unit, or may be a code or instructions executed by a controller/processor, e.g., controller 960. The code/instructions may be distributed among two or more controllers/processors.

Data recorder 908 may include a receiver or transceiver 944, a frame parser 970, and a controller or processor 990 for managing them. Processor 990 may be configured to carry out all or part of some embodiments of the present invention by for example executing software or code. Data recorder 908 may include additional components (e.g., USB interface, Secure Digital (“SD”) card driver/interface, controllers, etc.), elements or units for communicating with (e.g., transferring data frames, data, etc. to) a processing and/or displaying systems that may be configured to process images originating from in-vivo imager 912, localization data, and related data.

Transceiver 944 may receive 942 a data frame corresponding to a particular captured image, and frame parser 970 may parse the data frame to extract the various data contained therein (e.g., image data, decimated image associated with the particular captured image, localization data, etc.). In some embodiments, some data frames, which are referred to herein as “localization frames”, may be dedicated to contain and transfer only or mostly localization data. Localization frames may, for example, include localization data but not image data.

User workstation 930 may include a display or be functionally connected to one or more external displays, for example to display 902. Workstation 930 may receive frames (e.g., image frames, localization frames, etc.) or images from data recorder 908 and present them in real-time, for example as live video, or produce a video stream that also contains location and orientation information that may also be displayed on, for example, display 902. Workstation 930 may include a memory (e.g., memory 904) for storing frames transferred from data recorder 908 and possibly related metadata, and a processor (e.g., processor 905) for processing the stored frames and related data. Workstation 930 may display selected images or a video clip (e.g., a moving image stream) compiled from such images, e.g., to a human operator, health care or caregiving person, physician, etc. Processor 905 may be configured to carry out all or part of embodiments of the present invention.

Data recorder 908 may send P&O information, which pertains to or is derived from a current P&O of in-vivo device 906, to a magnetic field maneuvering control system (e.g., to controller 840 of FIGS. 8A-8B). The magnetic field maneuvering control system (e.g., controller 840) may generate a MMP based on the P&O information it receives from the data recorder 908, and P&O data pertaining to a new, or desired, P&O. For example, the magnetic field maneuvering control system (e.g., controller 840) may compare the P&O data it receives from the data recorder 908 to P&O data pertaining to a new (e.g., desired or intended) P&O, and, based on the comparison result, determine the next MMP that is to be applied to the in-vivo device.

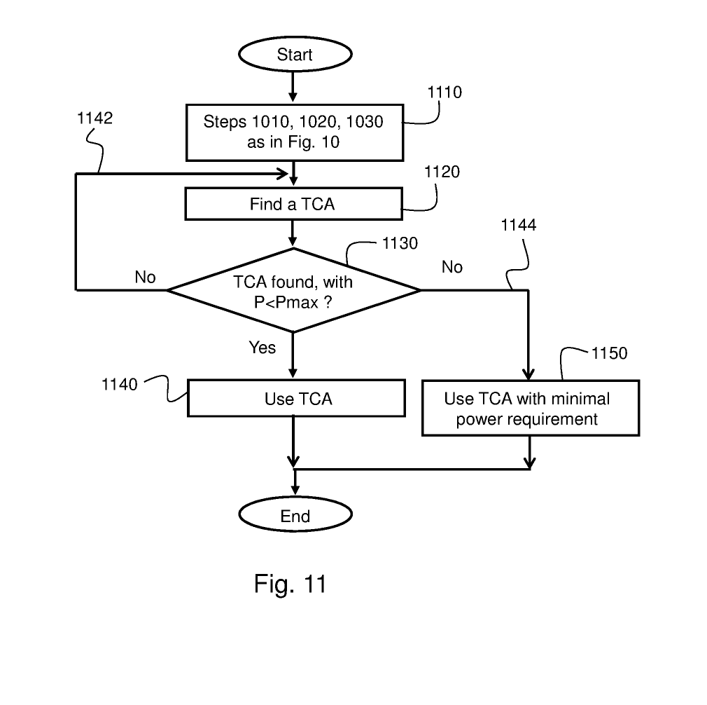

FIG. 10 shows a magnetically maneuvering method according to an example embodiment of the invention. As explained above, controller 840 may be configured to simultaneously, or substantially at the same time, generate an MMP whose magnetic field aligns an in-vivo device in a first direction, and a magnetic field gradient whose direction (e.g., that applies force on the in-vivo device) in a second direction that may differ from the first direction, to thereby manipulate the location and/or orientation of the in-vivo device. Controller 840 may be configured to select a set of TCAs in order to obtain the wanted or required MMP, as described below. Controller 840 may simultaneously, or substantially at the same time, generate the magnetic field and the magnetic field gradient to retain a location and/or orientation of the in-vivo device, or to move or rotate the in-vivo device to a wanted location and/or orientation. Controller 840 may select all TCAs to maneuver the in-vivo device, and individually calculate an electrical current for each coil of each TCA in order to generate an MMP that is required to maneuver the in-vivo device to the next location and, alternatively or additionally, to rotate it to the next orientation.

At step 1010, a magnetic maneuvering controller (e.g., controller 840 of FIGS. 8A-8B) may receive (current) P&O information of or pertaining to the current P&O of a maneuverable device (e.g., in-vivo device; e.g., in-vivo device 860 or 906). A MMP may be determined with respect to a spatial operating region that contains, or that is confined to a limited space that contains and encompasses, the device. Therefore, the current P&O of the device has to be known before a new MMP is calculated or otherwise determined. At step 1020, a new P&O may be determined/set (e.g., automatically; e.g., by the controller, or by a user) for the device.

At step 1030, the controller may determine a MMP) that is required to maneuver the device to the next location and/or orientation. There may be cases where a navigation/steering accuracy can be compromised. For example, when the in-vivo device is in the stomach, small deviations or inaccuracies in the location and/or orientation of the device may be acceptable or tolerated. However, when the device is in the small intestine, deviations tolerated in the stomach (for example) may not be tolerated in the small intestine. Therefore, permitted P&O tolerances/margins may be calculated, or otherwise determined, and stored (e.g., in a memory in system 805) in advance for various locations and orientations along and in the GI system. Then, the controller (e.g., controller 840) may calculate a permissible MMP range to maneuver the device to a next location and/or orientation, where the MMP range factors in the P&O accuracy permitted for that location and/or orientation. A permissible MMP range may refer to, or include, a permissible range of magnetic field strengths, a permissible range of magnetic field gradients, a permissible range of magnetic field directions, and a permissible range of magnetic field gradient directions.

At step 1040, the controller may select one or more MMPs within the permissible range of MMPs, and determine one or more potential sets of TCAs that may be suitable to generate the selected MMP(s). At step 1050, the controller may execute an optimization procedure in order to determine which set of TCAs, if used, would result in optimal power consumption.

The optimization process may be based on, or include use of, formula (1):

P=Σᵢ₌₁ⁿIᵢ²×Rᵢ (1)

where P is the overall electrical power expected to be consumed by the TCAs selected for participating in the generation of the MMP, n is the total number of coils participating in the generation of the MMP, Ii is the electrical current flowing through coil i (where i=1, 2, . . . n), and Ri is the electrical resistance of coil i.

The set of TCAs actually used to generate the MMP may be those for which P is minimal. Nevertheless, in some cases a less optimal set of TCAs (and electrical currents set) may be selected, for example in order to obtain a smoother transition of the maneuvered device from one position/orientation to another. The selection of TCAs and their electrical currents can be optimized because, while Maxwell's equations provide for eight degrees of freedom (DOF), the number of coils of the N TCAs, which may be equal to 3×N+4, provides for more DOF. For example, if N=8 and there are four Z-coils (as exemplified above), then there would be 3×8+4=28 coils.

### Finding a Currents' Set that Generates the Desired MMP (e.g., Field/Force/Torque) with Minimal Power Consumption

The magnetic field \vec{B}=[Bₓ B_{y} B_{z}]^{T} is to be generated by the TCAs (and by the additional ‘Z’ coils) described herein, and may be a function of the position \vec{P}=[x y z]^{T} of the in-vivo device in the gantry supporting, including or accomodating the coils, and of the current set \vec{I}=[I₁ I₂ . . . I_{N}]^{T}, where N is the number of independent (magnet) coils.

The magnetic field \vec{B} at any position \vec{P} within the operating region may be related to, or driven from, a (pseudo linear) superposition of the coils' currents:

$$\vec{B} ( \vec{P} , {[ \begin{matrix}{I}_{1} & {I}_{2} & … & {I}_{N}\end{matrix} ]}^{T} ) = {\sum }_{n = 1}^{N} {f}_{n} ( {I}_{n} ) \vec{B} ( P , \vec{{1}_{n} )}$$

where fₙ(Iₙ) is the non-linear gain function of iron core #n (if exists), and \vec{1ₙ} is a vector of all zeros, except for Iₙ which equals 1 A.

Since the magnetic torque \vec{T} and magnetic force \vec{F} at a given position and orientation are linear functions of the magnetic field \vec{B}, they are also (pseudo) linear over \vec{I}. So, in general, a vector \vec{ν}, which consists of a combination of elements of \vec{B}, \vec{F}, \vec{T} at a certain position \vec{P} and orientation R̅, can be written as:

$$\vec{v} ( \vec{P} , \overline{\overline{R}} , {[ \begin{matrix}{I}_{1} & {I}_{2} & … & {I}_{N}\end{matrix} ]}^{T} ) = {\sum }_{n = 1}^{N} {f}_{n} ( {I}_{n} ) \vec{v} ( \vec{P} , \overline{\overline{R}} , \vec{{1}_{n}} )$$

Therefore, the problem of finding the current set \vec{I} that achieves the desired magnetic field/force/torque vector \vec{ν} may be solved by first solving the linear equation system M \vec{I}ₗᵢₙₑₐᵣ=\vec{ν} (which is described in the next paragraph), and then calculating Iₙ=fₙ⁻¹(Iₗᵢₙₑₐᵣ_{ₙ}).
M={m_{k,n}} is a K by N matrix, where K is the length of the target vector v, and N is the number of independent coils. m_{k,n}=νₖ(\vec{P}, \vec{R}, \vec{1}ₙ). Since the number of linearly independent coils is greater than the number of the linearly independent elements of \vec{ν}, this equation set is underdetermined and its solution has N−K degrees of freedom (assuming that all coils are linearly independent and all elements of \vec{ν} are linearly independent). An electrical current solution that minimizes the required power may be found in the way in the non-limiting example shown below:
1. Find a particular solution to the problem by calculating the pseudo-inverse of M:

\vec{I}ₚ=M^{T}(MM^{T})⁻¹\vec{ν}
2. Find the null space (kernel) of M (i.e. NS_{[N×(N-K)]} such that M·NS·\vec{x}=0 for any \vec{x}_{[(N-k)×1]}):

NS=ker(M)
3. Find \vec{x} that minimizes the overall power. This may be a weighted least square problem, with a known solution (W=w_{i,i} is a diagonal weight matrix, where w_{i,i} is the resistance of coil #i):

\vec{x}=−(NS^{T}·W·NS)⁻¹NS^{T}·W·\vec{Iₚ}
4. The final solution will take the form \vec{Iₗᵢₙₑₐᵣ}=\vec{Iₚ}+NS·\vec{x}

In case the solution (resulting current) exceeds the current limit of an individual coil, or the total power exceeds the total power limit, the linear solution may be scaled down (i.e. \vec{Iₗᵢₙₑₐᵣ}=α\vec{Iₗᵢₙₑₐᵣ}, where αε(0,1)), until the current constraints and total power constraints are met, thus preserving the direction of \vec{ν} while reducing its magnitude.

Finally, the solution may be given by using the inverse gain function of the iron cores (if exist):

Iₙ=fₙ⁻¹(Iₗᵢₙₑₐᵣ_{ₙ})

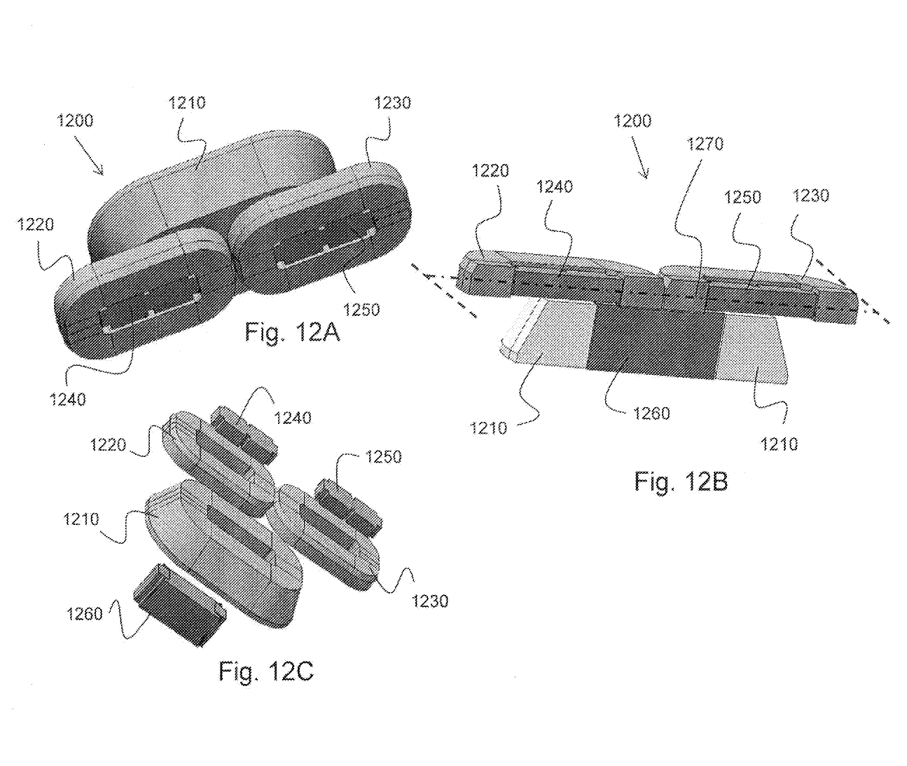

FIG. 11 shows a magnetically maneuvering method according to another example embodiment of the invention. Step 1110 may include the steps 1010, 1020 and 1030 described above in connection with FIG. 10.

Step 1120 may be similar to step 1040 of FIG. 10, except that step 1120 may results in one TCA setup. At step 1130, it is determined (e.g., by controller 840) whether a TCA setup has been found, for which the electrical power, P, dissipated on, or consumed by, the corresponding coils is less (lower) than a threshold value, Pmax. If a TCA setup has been found (shown as “Yes” at step 1130) (e.g., by controller 840), no other TCA setups are searched for (e.g., by controller 840), and the TCA setup found is used to generate the MMP (e.g., by controller 840), at step 1140. However, if a TCA setup has been found for which the electrical power, P, is greater (higher) than the threshold value, Pmax (shown as “No” at step 1130), another TCA setup may be searched for (per iteration loop 1142) at step 1120 (e.g., by controller 840). However, if a group of TCA setups have been found but none of the TCA setups satisfies condition 1130, a controller (e.g., controller 840) may use the TCA setup requiring the minimal electrical power (per branch 1144), as shown at step 1150.

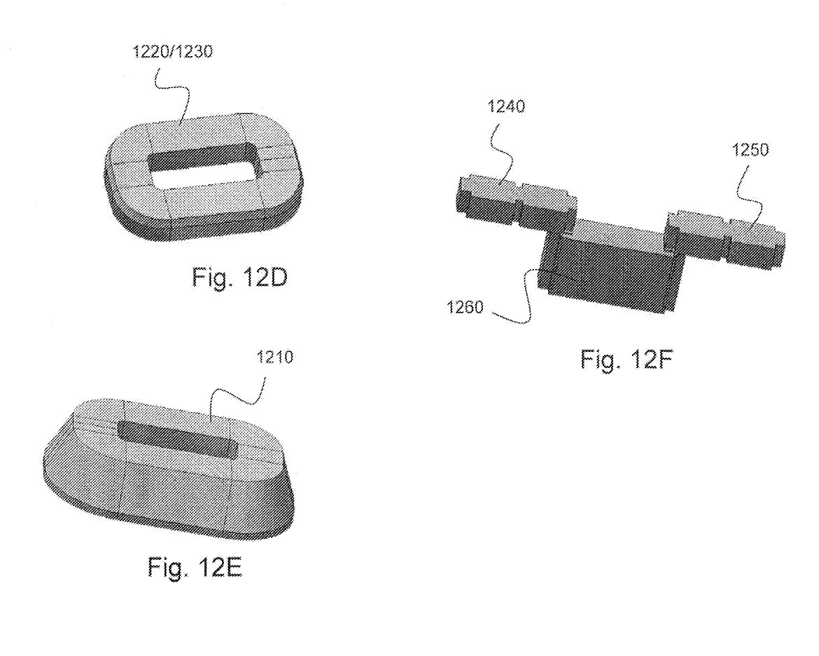

FIGS. 12A-12F depict a TCA according to another example embodiment of the present invention. FIG. 12A depicts an assembled TCA 1200. TCA 1200 may include an anterior coil 1220, a posterior coil 1230, and ancillary coil 1210. Coils 1220 and 1230 may adjacently be positioned side by side and may form or define a plane. (The plane is shown at 1270 in FIG. 12B.) Ancillary coil 1210 is attached to coils 1220 and 1230 such that coil 1210 partly overlaps coil 1220 and partly overlaps coil 1230. Each coil may have a magnetic core. For example, coil 1210 may have a core 1260, coil 1220 may have a core 1240 and coil 1230 may have a core 1250, as shown, for example, in FIGS. 12B and 12C.

FIG. 12B depicts a cross-sectional view of TCA 1200. FIG. 12C depicts an exploded view of TCA 1200. FIG. 12D depicts anterior coil 1220, which may be identical or similar to posterior coil 1230. FIG. 12E depicts ancillary coil 1210. FIG. 12F depicts the TCA's three magnetic cores 1240, 1250 and 1260.

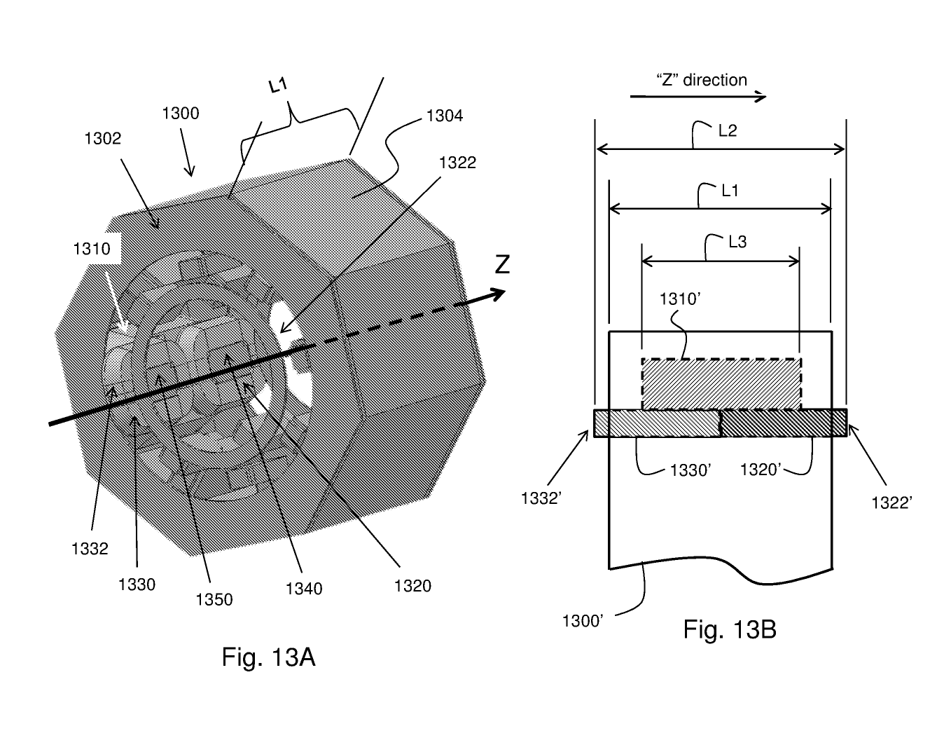

FIG. 13A depicts a magnetic system 1300 according to another example embodiment of the invention. Magnetic system 1300 may include N TCAs (for example eight TCAs), and a housing 1304 that houses the TCAs. One TCA is shown in full; i.e., the TCA including an anterior coil 1320, a posterior coil 1330, and an ancillary coil 1310 (part of coil 1310 is not shown because of coils 1320 and 1330 and their respective cores 1340 and 1350). Coils 1320 and 1330 are positioned side by side and form or define a plane similar to plane 1270 of FIG. 12B. Ancillary coil 1310 is attached to coils 1320 and 1330 such that ancillary coil 1310 partly overlaps anterior coil 1320 and partly overlaps posterior coil 1330. (For clarity of FIG. 13A, only magnetic cores of the other TCAs are shown.)

The coils setup (magnetic system) of FIG. 7B where all the TCAs are fully enclosed/housed by housing (gantry) 794 (the housing is sized to completely house/enclose all the TCAs), has a drawback, which is degraded magnetic characteristics (e.g., less magnetic field intensity in the center of the shell) due to end conditions of the housing enclosing the TCAs. FIG. 13A depicts a magnetic system that mitigates that problem: anterior coil 1320 (and also the anterior coils of the other N−1 TCAs) and posterior coil 1330 (and also the posterior coils of the other N−1 TCAs) each has a coil end (at 1322 and 1332, respectively) that extends from a side wall 1302 of housing 1304. Coil end 1332 extends from side wall 1302 of housing 1304. (Only coil end 1332 is visible but the same applies to the opposite end pointed at by arrow 1322).

The extending portions of coil ends 1322 and 1332 magnetically compensate for, or prevent, a magnetic field distortion due to the end conditions of housing 1304 and its side walls (e.g., side wall 1302). A drawing illustrating coil end extensions more clearly is shown in FIG. 13B. Referring to FIG. 13B, L1 (also shown in FIG. 13A) denotes the width of a coils house 1300′ (and of housing 1300); reference numerals 1310′, 1320′ and 1330′ are, respectively, an ancillary coil, an anterior coil and a posterior coil, which respectively correspond to coils 1310, 1320 and 1330 of FIG. 13A. Reference numerals 1322′ and 1332′ are respectively coil ends of coils 1320′ and 1330′, which respectively correspond to coil ends 1322 and 1332 in FIG. 13A. L2 is the overall length of coils 1320′ and 1330′ (and the overall length of coils 1320 and 1330 of FIG. 13A) when the anterior and posterior coils are put side by side (e.g., as shown in FIG. 12A). L3 is the length of ancillary coil 1330′ (and of ancillary coil 1330), where L2>L1>L3. Example values of L1, L2 and L3 are L1=730 mm, L2=890 mm (445×2) and L3=700 mm. In other words, using TCAs which are dimensioned such that the anterior coil and conjugated posterior coil of each TCA, when put adjacently, have an overall length L2 that meets the (‘compensation’ or ‘mitigation’) condition L2>L1, mitigates magnetic problems caused by end conditions of the housing that houses the TCAs.

Although embodiments of the invention are not limited in this regard, discussions utilizing terms such as, for example, “processing,” “computing,” “calculating,” “determining,” “inferring”, “deducing”, “establishing”, “analyzing”, “checking”, or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information non-transitory storage medium that may store instructions to perform operations and/or processes. Unless explicitly stated, the method embodiments described herein are not constrained to a particular order or sequence of steps, operations or procedures. Additionally, some of the described method embodiments or elements thereof can occur or be performed at the same point in time.

The articles “a” and “an” are used herein to refer to one or to more than one (e.g., to at least one) of the grammatical object of the article, depending on the context. By way of example, depending on the context, “an element” can mean one element or more than one element. The term “including” is used herein to mean, and is used interchangeably with, the phrase “including but not limited to”. The terms “or” and “and” are used herein to mean, and are used interchangeably with, the term “and/or,” unless context clearly indicates otherwise. The term “such as” is used herein to mean, and is used interchangeably, with the phrase “such as but not limited to”.

Different embodiments are disclosed herein. Features of certain embodiments may be combined with features of other embodiments; thus certain embodiments may be combinations of features of other or multiple embodiments. Embodiments of the invention may include an article such as a computer or processor non-transitory storage medium, such as for example a memory, a disk drive, or a USB flash memory, encoding, including or storing instructions, e.g., computer-executable instructions, which when executed by a processor or controller, carry out methods disclosed herein. For example, a system may include a non-transitory storage medium such as storage unit 940 and a controller such as controller 840. Some embodiments may be provided in a computer program product that may include a non-transitory machine-readable medium, having stored thereon instructions, which may be used to program a computer, or other programmable devices, to perform methods as disclosed above. Having thus described exemplary embodiments of the invention, it will be apparent to those skilled in the art that modifications of the disclosed embodiments will be within the scope of the invention. Alternative embodiments may, accordingly, include more modules, fewer modules and/or functionally equivalent modules. The present disclosure is relevant to various types of in-vivo devices (e.g., in-vivo devices with one or more imagers, in-vivo devices with no imagers at all, etc.), and to various types of receivers. Hence the scope of the claims that follow is not limited by the disclosure herein.

1. A magnetic system for maneuvering an in-vivo device in an operating region, the magnetic system comprising:
a number N of ternary coil assemblies, each ternary coil assembly comprising:
an anterior coil and a conjugated posterior coil, the anterior and posterior coils adjacently mounted side by side, the anterior and posterior coils respectively having an anterior axis and a posterior axis parallel to said anterior axis, each of the anterior and posterior coils configured to individually generate a magnetic field in a direction aligned with the respective axis and, jointly, in a direction which is at angle with respect to the axes; and
an ancillary coil attached to, or encircling the, anterior and posterior coils and having an ancillary axis parallel to, and in-between, the anterior and posterior axes,
wherein the N ternary coil assemblies are positioned to form a circle having a plane such that the anterior coils are positioned on one side of the plane and the posterior coils are positioned on the opposite side of the plane, and wherein each ternary coil assembly has a paired, conjugated, ternary coil assembly on the opposite side of the circle formed by the N ternary coils assemblies, and the anterior, posterior and ancillary axes of each ternary coil assembly are parallel to the plane.

2. The magnetic system as in claim 1, further comprising a controller to control operation of the N ternary coil assemblies to generate a magnetic field maneuvering pattern within the operating region.

3. The magnetic system as in claim 2, wherein the controller is configured to operate the N ternary coil assemblies to generate the magnetic field maneuvering pattern at a location of the in-vivo device, such that a magnetic field of the maneuvering pattern is in a first direction and a gradient of the magnetic field is in a second direction.

4. The magnetic system as in claim 2, wherein the controller is configured to selectively activate coil(s), for one or more ternary coil assemblies, which is/are selected from the group consisting of: anterior coil, posterior coil and ancillary coil, to generate the magnetic field maneuvering pattern.

5. The magnetic system as in claim 3, wherein the second direction is parallel to, or at angle alpha (a) with respect to, the first direction.

6. The magnetic system as in claim 5, wherein alpha (α) has a value within the range of 0 degrees to 90 degrees.

7. The magnetic system as in claim 5, wherein the first direction is parallel to, or perpendicular to, or at an angle beta (β) with respect to the normal of the plane of the N ternary coil assemblies.

8. The magnetic system as in claim 2, wherein the controller is configured to activate two or more pairs of ternary coil assemblies in order to change the magnetic field maneuvering pattern.

9. The magnetic system as in claim 2, further comprising a position and orientation system for providing data related to a current position and orientation of the in-vivo device in the gastrointestinal system, wherein the controller is further configured to control the N ternary coil assemblies based on the position and orientation data and an intended position and/or orientation of the in-vivo device.

10. The magnetic system as in claim 1, further comprising one or more parallel annularly-shaped coils for generating magnetic fields in a direction of the normal of the plane of the N ternary coil assemblies.

11. The magnetic system as in claim 1, wherein N is an even number.

12. The magnetic system as in claim 1, wherein N is equal to eight.

13. The magnetic system as in claim 1, wherein the ancillary coil of the ternary coil assembly is configured to generate an ancillary magnetic field in a direction of the ancillary axis.

14. The magnetic system as in claim 1, further comprising a housing to house the N ternary coil assemblies, said housing having a width L1, wherein the anterior coil and the conjugated posterior coil of each ternary coil assembly have an overall length L2, where the value of L2 is greater than the value of L1.

15. A ternary coil assembly for a magnetic system for maneuvering an in-vivo device, comprising,
an anterior coil;
a posterior coil adjacently mounted side by side with respect to, and electrically isolated from and forming a plane with, the anterior coil; and
an ancillary coil attached to or encircling, and electrically isolated from, the anterior and posterior coils,
wherein the anterior and posterior coils are configured to, by manipulating their electrical current, generate a magnetic field in a first direction and a magnetic field gradient in a second direction different from the first direction.

16. The ternary coil assembly as in claim 15, wherein the anterior coil, the posterior coil and the ancillary coil respectively have an anterior axis, a posterior axis and an ancillary axis, wherein the anterior, posterior and ancillary axes are mutually parallel, and wherein the ancillary axis is between the anterior axis and the posterior axis.

17. A method for magnetically maneuvering an in-vivo device in a gastrointestinal system, comprising:
simultaneously generating a magnetic field to align an in-vivo device in a first direction and a magnetic field gradient to exert force on the in-vivo device in a second direction to thereby manipulate the location and/or orientation of the in-vivo device.

18. The method as in claim 17, wherein simultaneously generating the magnetic field and the magnetic field gradient is to retain a current location and/or orientation of the in-vivo device, or to move or rotate the in-vivo device to a wanted location and/or orientation.

19. The method as in claim 17, wherein the first and second directions are at angle alpha (α) with respect to each other.

20. The method as in claim 19, wherein alpha (α) has a value between 0 degrees and 90 degrees.

21. A method of operating a magnetic system for maneuvering an in-vivo device in a gastrointestinal system, comprising:
in a magnetic maneuvering system comprising:
a number N of ternary coil assemblies, where each ternary coil assembly comprises,
an anterior coil and a posterior coil, the anterior and posterior coils adjacently mounted side by side, the anterior and posterior coils respectively having an anterior axis and a posterior axis parallel to said anterior axis, each of the anterior and posterior coils configured to individually generate a magnetic field in a direction aligned with the respective axis and, jointly, in a direction perpendicular to the axes;
an ancillary coil, said ancillary coil encircling the anterior and posterior coils and having an ancillary axis parallel to, and in-between, the anterior and posterior axes,
wherein the N ternary coil assemblies are positioned circularly and form a plane such that the anterior and posterior coils are positioned in opposite directions with respect to the plane, and wherein each ternary coil assembly has a paired ternary coil assembly on the opposite side of the circle, and the anterior, posterior and ancillary axes of each ternary coil assembly are perpendicular to a normal of the plane; and
a controller configured to operate the N ternary coil assemblies to generate a magnetic field maneuvering pattern within an operating region,
the method comprising:
receiving, by the controller, a location and orientation data representative of a current location and orientation of an in-vivo device in the gastrointestinal system of a subject positioned in the circular N ternary coil assemblies;
obtaining, by the controller, an intended location and/or orientation of the in-vivo device in the gastrointestinal system; and
generating, by the controller, a control signal for each ternary coil assembly based on the location and orientation data and on the intended location and orientation.

22. The method as in claim 21, wherein generating the control signal for each ternary coil assembly comprises:
calculating one or more sets of coil currents for the N ternary coil assemblies; and
selecting for operation a set of coil currents that results in a minimum electrical power consumption.
